(19) United States
(12) Patent Application Publication
(10) Pub. No.: US 2026/0215767 A1
(43) Pub. Date: Jul. 30, 2026

(54) HIGH SHAPE-RETENTION RETRACTOR

(71) Applicants: UNIVERSITY PUBLIC CORPORATION OSAKA, Osaka-shi, Osaka (JP); OZK CO., LTD., Yao-shi, Osaka (JP); SHINWA SYOJI CO., LTD., Higashiosaka-shi, Osaka (JP)

(72) Inventors: Toshihiko SHIBATA, Osaka-shi, Osaka (JP); Yosuke TAKAHASHI, Osaka-shi, Osaka (JP); Haruhiko YAMASAKI, Yao-shi, Osaka (JP); Katsunori MITSUHASHI, Yao-shi, Osaka (JP); Yoshiro MORISHITA, Higashiosaka-shi, Osaka (JP)

(21) Appl. No.: 19/145,821

(22) PCT Filed: Jan. 29, 2024
(86) PCT No.: PCT/JP2024/002539

§ 371 Date: Jul. 3, 2025

(30) Foreign Application Priority Data
Jan. 31, 2023 (JP) 2023-013557

(51) Int. Cl. A61B 17/02

(52) U.S. Cl. CPC A61B 17/02; A61B 2017/0237

(57) ABSTRACT

There is provided a retractor for holding an incision site of the human body to be incised in an open state, the retractor being a high shape-retention retractor whose shape during use is stabilized. The high shape-retention retractor: contains, as a main resin component, a synthetic resin having chemical resistance and being an elastic material; and is configured to be elastically curved from a plate state such that a first end and a second end overlap in a thickness direction to form a ring, in which: a through-hole is formed near the first end to allow the second end to pass through; and a size of a diameter of the ring can be varied by changing a length of a distance over which the second end is passed through the through-hole.

### TECHNICAL FIELD

The present invention relates to a retractor, and particularly to a high shape-retention retractor for holding an incision site of the human body to be incised in an open state in a surgery in which a surgeon directly performs the surgery by a technique or a so-called robot surgery in which a surgeon performs the surgery by manipulating a robot.

### BACKGROUND ART

Conventionally, there is a retractor in which a thin plate formed of an elastic material is inserted into an incision site in a rolled-up state, and when the hands with which the thin plate is rolled-up are released after the insertion, the thin plate expands by itself by the elastic force of the thin plate, and the diameter of the cylinder can be increased (Patent Literature 1). Among such conventional retractors, the retractor illustrated in FIG. 32A and FIG. 32B does not have an accessory other than the thin plate, and thus there is no risk that an accessory may be caught on a region of the human body or the like during inserted into an incision site.

Since such a retractor holds the incision site in an open state by its own elastic force, the necessity of pulling the incision site by hooks is reduced. And, the human body is less likely to be injured by the hooks.

### CITATION LIST

### Patent Literature

Patent Literature 1: JP 2007-82674 A

### SUMMARY OF INVENTION

### Technical Problems

Perspective views of the conventional retractor are illustrated in FIG. 32A and FIG. 32B. As in FIG. 32B that illustrates a state during use, the retractor has a cylindrical shape with the same diameter from top to bottom. An instrument held by the fingers of a surgeon or an instrument held by a robot arm during surgery enters from the upper opening of the cylinder, and administers treatment to the affected area located in the lower opening of the cylinder.

At this time, contact between the instrument and the upper edge of the cylinder may cause damage to the retractor, or lead to situations where the retractor becomes tilted or the overlapping areas of the retractor become misaligned. In addition, it is difficult to tilt and hold the instrument in the horizontal direction without touching the cylinder in order to allow the tip of the instrument to reach the affected area.

In addition, when the expansion by the elastic force proceeds, there is a large risk that both ends that should originally overlap may be separated from each other.

In view of these problems, an object of the present invention is to provide a retractor whose shape during use is stabilized.

### Solutions to Problems

(1) The present invention provides a high shape-retention retractor that: contains, as a main resin component, a synthetic resin having chemical resistance and being an elastic material; has a first end and a second end separated from the first end in a state of being developed in a plate shape, and has a width direction; and is configured to be elastically curved from the plate state such that the first end and the second end overlap in a thickness direction of the plate state to form a ring, in which: a through-hole is formed to allow the second end to pass through in a first end nearby area that is near the first end; and a size of a diameter of the ring can be varied by changing a length of a distance over which the second end is passed through the through-hole.

That is, the high shape-retention retractor of the present invention is configured to be curved from a plate state to form a ring, and does not form a cylindrical shape as conventional retractors do. Therefore, there is little risk that a surgical instrument may cause damage or a contact with an instrument may make the shape unstable. Furthermore, a wide field of view for observing the affected area can be secured.

In addition, the high shape-retention retractor of the present invention contains, as a main resin component, a synthetic resin having chemical resistance, and thus it can be cleaned using a chemical product every time it is used for surgery and can be repeatedly used. In addition, by containing the synthetic resin, it is easy to mold into a thin plate shape.

Furthermore, the high shape-retention retractor of the present invention has the through-hole formed in the first end nearby area, and thus there is little risk that in a state where the second end is passed through the through-hole, the first end side and the second end side may be separated from each other. Therefore, the shape during use is stabilized. In addition, the first end side is reliably in contact with the second end side, and thus a frictional force is generated between them. Therefore, by reducing the risk that the ring of the high shape-retention retractor of the present invention may rapidly expand or rapidly contract, the shape during use can also be stabilized.

(2) In addition, a second end nearby area that is near the second end may have an engaged portion protruding from the through-hole in the width direction in a state where the second end is passed through the first end nearby area.

That is, even if the high shape-retention retractor forming the ring is about to expand, the engaged portion hooks onto the periphery of the through-hole, and thus the second end nearby area is engaged by the first end nearby area in a direction that tends to contract the ring, whereby further expansion can be prevented. As a result, the shape during use can be stabilized.

(3) In addition, a pair of the engaged portions may be provided, the respective engaged portions may protrude in both outward directions in the width direction, and at least the engaged portion provided on one side in the width direction may be formed to protrude from a constricted portion adjacent in a direction coming close to the first end and recessed inward in the width direction.

That is, the pair of the engaged portions protruding in both directions in the width direction is provided, and thus each engaged portion hooks onto the periphery of the through-hole, so that the second end nearby area is efficiently engaged by the first end nearby area.

In addition, the constricted portion is provided and the engaged portion on one side protrudes from the constricted portion, and thus the engaged portion on the other side, which is narrowed by the constricted portion, can pass through the through-hole even if the width of the through-hole is small. By reducing the width of the through-hole in this manner, the engaged portion easily hooks onto the periphery of the through-hole, and the second end nearby area is more efficiently engaged by the first end nearby area.

(4) In addition, an inner peripheral edge of the through-hole may have a shape having a line segment between two points, and the line segment between the two points may be inclined such that, of the two points, a first point located closer to a first edge in the width direction comes closer to either the first end or the second end than a second point located closer to a second edge in the width direction does.

When the high shape-retention retractor in a rolled-up state fits in a region like an internal organ, muscle, or the like surrounding the periphery of the affected area (hereinafter, a region like an internal organ, muscle, or the like surrounding the periphery of the affected area is simply referred to as a “region such as surrounding visceral organs”), the region such as surrounding visceral organs does not necessarily have a columnar space having the same cross section such as a cylinder shape or a quadrangular prism shape. In some cases, the space of the region, such as surrounding visceral organs, is partially or entirely formed in a shape close to a frustum shape such as a truncated cone shape whose diameter decreases or increases toward the back.

In the high shape-retention retractor of the present invention, the line segment of the through-hole is inclined, and thus when the second end passes through the through-hole in the direction orthogonal to the inclined line segment, the extension lines of the respective generatrices at different positions from the first end to the second end intersect with or come close to each other, and the outward-facing surface forms a part of the side surface of a substantially truncated cone. For example, if the length of the through-hole in the inclination direction of the line segment is only slightly larger than the width, on the second end side, of the high shape-retention retractor, the second end easily passes through the through-hole in the direction orthogonal to the inclination direction, whereas it is likely to be caught on the periphery of the through-hole and difficult to pass through the through-hole in a direction not orthogonal to the inclination direction. Therefore, with the high shape-retention retractor of the present invention, the diameter can be slightly varied in a state where the outward-facing surface forms a part of the side surface of the substantially truncated cone by the inclination of the line segment of the through-hole. As a result, it is easy for the outward-facing surface to follow the shape of a part or the whole of the space of the region such as surrounding visceral organs, and the retractor fits well after being inserted into the incision site.

(5) In addition, in a central area that is an area sandwiched between the first end nearby area and the second end nearby area, a center line in the width direction may draw an arc in the developed state.

That is, the center line of the central area draws an arc, and thus the high shape-retention retractor of the present invention forms a curved surface shape by being curved from a plate state to form a ring, and its outward-facing surface forms the side surface of the substantially truncated cone. In addition, the inward-facing surface forms the back surface of the side surface. At this time, a curved surface is formed in which a ½ area on the inner side of the center line that is an arc is inclined so as to form the ½ side surface on the small-diameter side of the substantially truncated cone. Similarly, a curved surface is formed in which a ½ area on the outer side of the center line is inclined so as to form the ½ side surface on the large-diameter side of the substantially truncated cone.

As a result, in the high shape-retention retractor of the present invention in a state where the retractor is curved to form a ring, it is easy for the outward-facing surface to follow the shape of a part or the whole of the space of the region such as surrounding visceral organs, and the retractor fits well after being inserted into the incision site.

(6) In addition, the inner peripheral edge of the through-hole may have a shape having a line segment between two points, and the line segment between the two points may be inclined with respect to the width direction such that, in the developed state, an extension line, extended inward of the arc, of the line segment between the two points passes through a position farther from the arc than a center point of the arc.

The inventors have found that the center line of the high shape-retention retractor of the present invention draws an arc, and thus a three-dimensional shape after winding tends to form a spiral shape by the elastic force when the retractor is curved from a plate shape to form a ring. That is, if the second end is not passed through the through-hole, an area close to the first end and an area close to the second end, after winding, are misaligned in the axial direction of the truncated cone, which tends to form a spiral shape. In addition, it has been found that when the second end of such a high shape-retention retractor is passed through the through-hole, the area close to the second end tends to protrude toward the large-diameter side of the truncated cone, and as a result, does not overlap so as to coincide with the area close to the first end one turn earlier. Thus, a force that tends to bias the second end toward the large-diameter side of the truncated cone is applied to the high shape-retention retractor, so that, under the condition, the outward-facing surface and inward-facing surface of the high shape-retention retractor may form the front surface and back surface of a distorted truncated cone, respectively.

On the other hand, the through-hole of the high shape-retention retractor of the present invention is inclined such that the extension line, extended inward of the arc, of the line segment passes through a position farther from the arc than the center line of the arc. Therefore, it has been found that the second end that has passed through the through-hole after making a full turn tends to advance closer to the small-diameter side of the truncated cone, which is the direction orthogonal to the inclination direction, than along the arc. That is, it has been found that the second end easily passes through the through-hole in the direction orthogonal to the inclination direction, whereas it is likely to be caught on the periphery of the through-hole and difficult to pass through the through-hole in a direction not orthogonal to the inclination direction. Therefore, a force that tends to bias the second end toward the small-diameter side, through which the second end easily passes, of the truncated cone is applied to the high shape-retention retractor.

As described above, in the high shape-retention retractor of the present invention, the center line draws an arc and the through-hole is inclined in a predetermined direction, and thus the force that tends to bias the second end toward the large-diameter side of the truncated cone can be balanced with the force that tends to bias it toward the small-diameter side thereof. As a result, the second end is less likely to come close to either the large-diameter side or the small-diameter side, and the outward-facing surface and inward-facing surface of the high shape-retention retractor can form the front surface and back surface of an undistorted truncated cone, respectively. Therefore, it is easy for the outward-facing surface to follow the shape of a part or the whole of the space of the region such as surrounding visceral organs, and the retractor fits well after being inserted into the incision site.

(7) In addition, a plate thickness in the thickness direction may be 0.1 mm or more and less than 0.5 mm.

The inventors have found through trial and error that even in a configuration in which such a ring is formed, the high shape-retention retractor of the present invention is less likely to injure the affected area and the shape of the ring is further stabilized when the plate thickness of the high shape-retention retractor is 0.1 mm or more and less than 0.5 mm. That is, it has been found that if the plate thickness is less than 0.1 mm, the edge of the thin plate, when placed at the incision site, becomes too sharp, leading to a high risk that the affected area or the area surrounding it may be injured. On the other hand, it has been found that if the plate thickness is 0.5 mm or more, the elastic force of the thin plate is too strong to maintain a shape in which the direction of the first end side and the direction of the second end side overlap in the state of coinciding with each other, and for example, a shape, in which the direction of the first end side does not coincide with the direction of the second end side and the first end and the second end are misaligned, may be created.

As a result, with the high shape-retention retractor of the present invention, the shape during use can be easily stabilized.

(8) In addition, the central area that is an area sandwiched between the first end nearby area and the second end nearby area may have a wavy portion where at least one edge in the width direction is wavy in the width direction.

That is, the high shape-retention retractor of the present invention has the wavy portion on at least one side edge, and thus when the high shape-retention retractor is inserted into the incision site such that the side edge having the wavy portion is located on the back side, the side edge having the wavy portion can be in contact with the affected area or its surroundings on the back side. At this time, the wavy portion is wavy, and thus the side edge having the wavy portion is less prone to slipping against the affected area or its surroundings. Therefore, even when the high shape-retention retractor forming the ring is about to further expand outward or contract inward, the rapid expansion or contraction can be prevented by a resistance force that the wavy portion receives from the affected area or its surroundings. This makes it possible to stabilize the shape of the high shape-retention retractor of the present invention in a state of being expanded to a certain extent.

(9) In addition, the central area that is an area sandwiched between the first end nearby area and the second end nearby area may have an uneven portion where at least one edge in the width direction is uneven in the width direction.

That is, the high shape-retention retractor of the present invention has the uneven portion on at least one side edge, and thus when the high shape-retention retractor is inserted into the incision site such that the side edge having the uneven portion is located on the back side, the side edge having the uneven portion can be in contact with the affected area or its surroundings on the back side. At this time, the uneven portion is uneven, and thus the side edge having the uneven portion is less prone to slipping against the affected area or its surroundings. Therefore, even when the high shape-retention retractor forming the ring is about to further expand outward or contract inward, the rapid expansion or contraction can be prevented by a resistance force that the uneven portion receives from the affected area or its surroundings. This makes it possible to stabilize the shape of the high shape-retention retractor of the present invention in a state of being expanded to a certain extent.

(10) In addition, a plurality of substantially rectangular quadrilateral portions, each having long sides in the width direction and recessed or projected in the thickness direction, may be provided in the central area that is an area sandwiched between the first end nearby area and the second end nearby area, and in a state where the ring is formed, each of the quadrilateral portions may be recessed radially inward or projected radially outward of the curve on an outer surface of the curve.

That is, the high shape-retention retractor of the present invention has the plurality of quadrilateral portions on the outer surface when curved, and thus when the high shape-retention retractor is inserted into the incision site, the quadrilateral portions can be in contact with the affected area or the region, such as surrounding visceral organs, on the back side. At this time, the quadrilateral portions are recessed into or projected from the outer surface, and thus the central area has a large friction with these portions and is less prone to slipping. Therefore, even when the annular high shape-retention retractor is about to expand or about to rotate or move, these can be prevented by the resistance force that the quadrilateral portions receive from the affected area or the region, such as surrounding visceral organs, on the back side. This makes it possible to stabilize the shape, posture, or position of the high shape-retention retractor of the present invention.

(11) In addition, a polyether ether ketone resin may be contained as the resin component.

That is, a polyether ether ketone resin (PEEK) has heat resistance in addition to chemical resistance, and thus the retractor can be cleaned using a liquid such as a high-temperature detergent or cleaning water. Therefore, more effective cleaning can be performed. In addition, effective cleaning can be performed without applying a large mechanical force, and thus there is little risk that the surface may be damaged during the cleaning. This makes it possible to repeatedly use the high shape-retention retractor.

Furthermore, a polyether ether ketone resin has impact resistance and abrasion resistance, and thus even if coming into contact with a metal instrument, the high shape-retention retractor of the present invention is less likely to be damaged. In addition, even if coming into contact with a part of a remotely operated instrument or robot in so-called robot surgery, the retractor is less likely to be damaged. These make it possible to use the high shape-retention retractor repeatedly over a long period of time. In addition, there is no risk that the damaged portion of the retractor may injure the human body such as an internal organ.

### Advantageous Effects of Invention

In the present invention, there is provided a high shape-retention retractor that maintains a stable shape during use, has a long service life, and poses no risk of injuring the human body, as described above. In addition, a high shape-retention retractor that can be easily manufactured is provided.

### BRIEF DESCRIPTION OF DRAWINGS

FIG. 1 is a view of a state where a high shape-retention retractor of a first embodiment of the present invention is unfolded.

FIG. 2 is a view created by tracing an image in which the high shape-retention retractor in FIG. 1 in its assembled state is viewed from slightly below the front side.

FIG. 3 is a photograph of a state where the high shape-retention retractor in FIG. 1 in its assembled state is placed at an area where the heart of an animal is incised.

FIG. 4 is a photograph showing a state where the high shape-retention retractor in FIG. 1 in its assembled state is placed inside the heart of an animal.

FIG. 5A is a view created by tracing an image in which the high shape-retention retractor in FIG. 1 in its assembled state is viewed from slightly below the rear side. FIG. 5B is a view created by tracing an image of a state where an insertion end of the high shape-retention retractor in FIG. 1 in its assembled state is inserted more deeply than in the state in FIG. 5A.

FIG. 6 is a front view of a state where a through-hole nearby portion and an engaged portion nearby portion of the high shape-retention retractor in FIG. 1 overlap in the front-rear direction.

FIG. 7 is a front view of a state where an insertion end of the high shape-retention retractor in FIG. 6 advances rightward.

FIG. 8A and FIG. 8B are views of states where a high shape-retention retractor is unfolded to illustrate a first modification and a second modification of the first embodiment of the present invention.

FIG. 9A and FIG. 9B are views of states where a high shape-retention retractor is unfolded to illustrate a third modification and a fourth modification of the first embodiment of the present invention.

FIG. 10A is a front view of a state where a high shape-retention retractor is unfolded to illustrate a fifth modification of the first embodiment of the present invention. FIG. 10B is a plan view of the state in FIG. 10A. FIG. 10C is a partially enlarged view of FIG. 10B.

FIG. 11A and FIG. 11B are schematic views for explaining a high shape-retention retractor of a second embodiment of the present invention.

FIG. 12A is a view of a state where the high shape-retention retractor of the second embodiment of the present invention is unfolded. FIG. 12B is a partially enlarged view of a through-hole of the high shape-retention retractor in FIG. 12A.

FIG. 13A and FIG. 13B are each front views of a state where an engaged portion nearby portion and the central portion of an area adjacent thereto of the high shape-retention retractor in FIG. 12 in its assembled state pass through the through-hole and overlap in the rear direction. FIG. 13A illustrates an orthogonal insertion state, while FIG. 13B illustrates an oblique insertion state.

FIG. 14 is a front view of a state where the engaged portion nearby portion is pulled from the state in FIG. 13A in the direction opposite to the insertion direction.

FIG. 15 is a view of a state where a high shape-retention retractor is unfolded to illustrate a modification of the second embodiment of the present invention.

FIG. 16 is a view of a state where a high shape-retention retractor of a third embodiment of the present invention is unfolded.

FIG. 17 is a partially enlarged view of a through-hole of the high shape-retention retractor in FIG. 16.

FIG. 18 is a view created by tracing an image in which a transparent model of the high shape-retention retractor in FIG. 16 is made and the model in an orthogonal insertion state is viewed from above the rear side.

FIG. 19 is a front view of a state where an engaged portion nearby portion is pulled from the state in FIG. 18 in the direction opposite to the insertion direction.

FIG. 20 is a view of a state where a high shape-retention retractor of a fourth embodiment of the present invention is unfolded.

FIG. 21 is a partially enlarged view of a through-hole of the high shape-retention retractor in FIG. 20.

FIG. 22 is a view created by tracing an image in which a transparent model of the high shape-retention retractor in FIG. 20 is made and the model in the orthogonal insertion state is viewed from above the rear side.

FIG. 23 is a front view of a state where the engaged portion nearby portion is pulled leftward from the state in FIG. 22.

FIG. 24 is a view of a state where a high shape-retention retractor of a fifth embodiment of the present invention is unfolded.

FIG. 25A is a perspective view in which the high shape-retention retractor in FIG. 24 in its assembled state is viewed from obliquely above the through-hole nearby portion. FIG. 25B is a front view of the high shape-retention retractor in FIG. 25A with the through-hole nearby portion viewed from the front.

FIG. 26 is a view of a state where a high shape-retention retractor of a sixth embodiment of the present invention is unfolded.

FIG. 27 is a partially enlarged view in which a through-hole nearby portion of the high shape-retention retractor in FIG. 26 is enlarged.

FIG. 28A is a perspective view in which the high shape-retention retractor in FIG. 26 in its assembled state is viewed from obliquely above the through-hole nearby portion. FIG. 28B is a front view of the high shape-retention retractor in FIG. 28A with the through-hole nearby portion viewed from the front.

FIG. 29A and FIG. 29B are views created by tracing an image in which the high shape-retention retractor in FIG. 24 in its assembled state is viewed from obliquely above the through-hole nearby portion. FIG. 29C is a view created by tracing an image in which the high shape-retention retractor in FIG. 29B is viewed from the front side.

FIG. 30A and FIG. 30B are views created by tracing an image in which the high shape-retention retractor in FIG. 26 in its assembled state is viewed from obliquely above the through-hole nearby portion. FIG. 30C is a view created by tracing an image in which the high shape-retention retractor in FIG. 30B is viewed from the front side.

FIG. 31 is a view of a state where a high shape-retention retractor of the present invention, having a shape in which the constricted portion is recessed adjacent to the second engaged portion, closer to the central portion, is unfolded.

FIG. 32A and FIG. 32B are perspective views of a conventional retractor.

### DESCRIPTION OF EMBODIMENTS

### First Embodiment

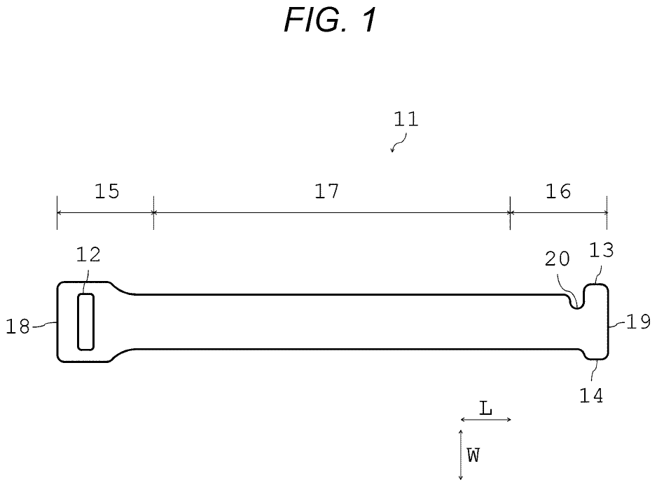

A first embodiment of the present invention will be exemplified with reference to FIG. 1 to FIG. 10. In FIG. 1, reference numeral 11 denotes a high shape-retention retractor. The high shape-retention retractor refers to an instrument that stabilizes the intraoperative shape and secures the surgical field in order to hold the incision site of the human body incised during surgery in an open state. The high shape-retention retractor 11 of the present invention is a strip-shaped thin plate in a state of being developed in a plate shape as illustrated in FIG. 1. A horizontal arrow L illustrated in FIG. 1 indicates the longitudinal direction of the high shape-retention retractor 11, while a vertical arrow W indicates the width direction thereof. Although not illustrated in FIG. 1, the high shape-retention retractor 11 has a thickness in the depth direction in the view.

The high shape-retention retractor 11 has a first end that is one end in the longitudinal direction and a second end that is the other end separated from the one end. The first end is located on the left side in the view, while the second end is located on the right side in the view. A through-hole 12 in the thickness direction is formed in the nearby area of the first end. In addition, a pair of engaged portions 13, 14 is provided in the nearby area of the second end. The respective engaged portions 13, 14 are provided so as to protrude toward both outward directions, respectively, in the width direction of the high shape-retention retractor 11. Of the pair of engaged portions 13, 14, the first engaged portion 13 is provided upward in FIG. 1, while the second engaged portion 14 is provided downward in FIG. 1. The first end is also referred to as an insertion receiving end 18, and the second end is also referred to as an insertion end 19. Furthermore, the nearby area of the first end is also simply referred to as a first end nearby area or a through-hole nearby portion 15, and the nearby area of the second end is also simply referred to as a second end nearby area or an engaged portion nearby portion 16.

A central area that is an area sandwiched between the through-hole nearby portion 15 and the engaged portion nearby portion 16 in the longitudinal direction is referred to as a central portion 17. The central portion 17 extends in the longitudinal direction, and both ends thereof in the width direction are parallel to each other.

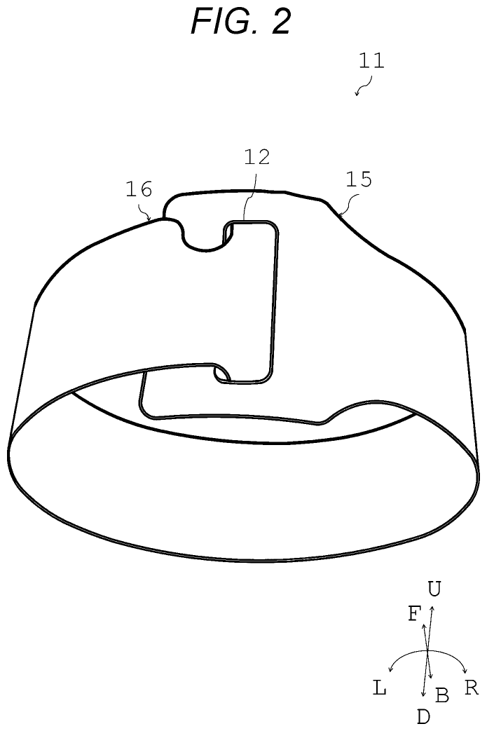

The high shape-retention retractor 11: is elastically curved from the plate state, so that the insertion receiving end 18 side and the insertion end 19 side overlap in the thickness direction of the plate state and are rolled up, whereby a ring that is a substantially annular ring can be formed. FIG. 2 is a view created by tracing an image in which the high shape-retention retractor 11 illustrated in FIG. 1 is made, the insertion end 19 is curved so as to be turned to the back side from the strip-shaped state, and then the insertion end 19 is passed through the through-hole 12 from the front side. That is, the insertion end 19 is passed through the through-hole 12 from the outer side to the inner side of the annular shape. The state in this view is referred to as an assembled state. The direction of an arrow U indicates the upper side in each view of the high shape-retention retractor 11 in its assembled state, and the direction of an arrow D indicates the lower side in each view thereof. The direction of an arrow L indicates the left side in each view thereof, and the direction of an arrow R indicates the right side in each view thereof. In addition, the direction of an arrow F indicates the front side in each view thereof, and the direction of an arrow B indicates the rear side in each view thereof. The outward-facing surface of the high shape-retention retractor 11 in its assembled state forms the side surface of a substantially disk-shaped body having a vertical centerline. However, each arrow U, D, L, R, F, B does not necessarily indicate the upper side, lower side, left side, right side, front side, and rear side, respectively, when the high shape-retention retractor 11 is used. Furthermore, the high shape-retention retractor 11 is not only assembled by being curved so as to turn the insertion end 19 to the back side as illustrated in FIG. 2, but also assembled by being curved so as to turn the insertion end 19 from the front side in some cases, and the assembled state in this case is illustrated in a laterally reversed manner. As described above, the high shape-retention retractor 11 is used such that the direction that is convenient for a user is set to the upper side, lower side, left side, right side, front side, and rear side, and is assembled and used such that whichever surface, front or back, which is more convenient to use, is exposed to the front surface.

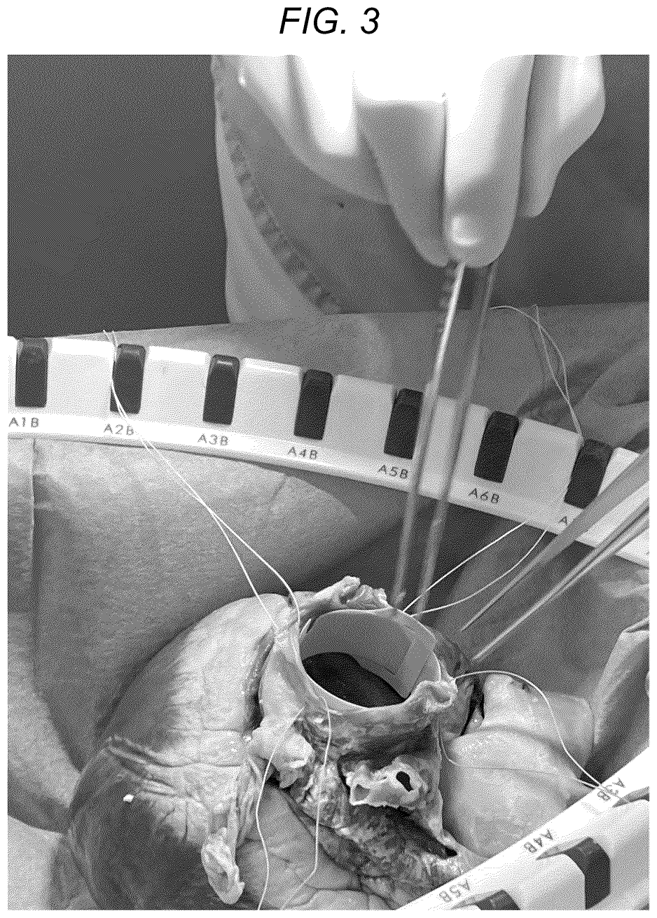

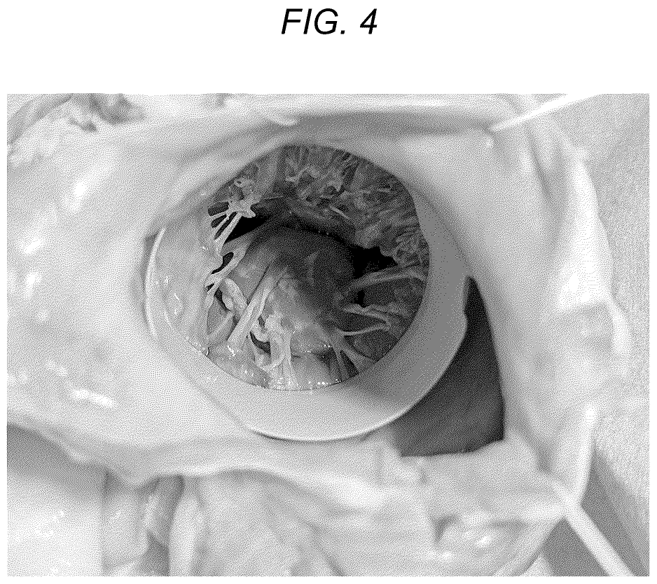

As an example, FIG. 3 is a photograph in which the high shape-retention retractor 11 in its assembled state is placed at an area where the heart of an animal is incised. The high shape-retention retractor 11 in FIG. 3 is used incidentally in the same back-front and up-down directions as those in FIG. 2. In the example of this photograph, a state is imaged in which the high shape-retention retractor 11 in its assembled state is fitted into the incision site of the heart, and a user, a surgeon, inserts an instrument held by fingers into the inside of the high shape-retention retractor 11 in its assembled state to treat the affected area inside the heart. The high shape-retention retractor 11 has elasticity and is assembled such that the insertion end 19 passes through the through-hole 12, and thus even if some force is applied from the outside or inside of the high shape-retention retractor 11 in its assembled state during placement, such force can be absorbed with the high shape-retention retractor 11 elastically deforming or the insertion end 19 sliding in the left-right direction with respect to the through-hole 12. As a result, the shape of the spread state as illustrated in FIG. 3 can be stabilized. FIG. 4 is a photograph taken by looking into the opening of the incision site to capture the high shape-retention retractor 11 placed to be in contact with the affected area or its surroundings on the back side.

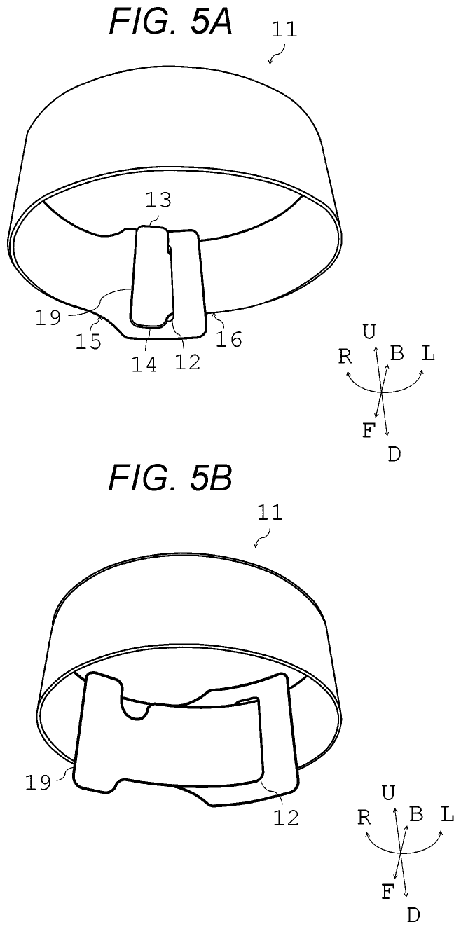

FIG. 2 is a view created by tracing an image in which the high shape-retention retractor 11 in its assembled state is viewed from slightly below the front side, whereas FIG. 5A and FIG. 5B are views each created by tracing an image in which the same is viewed from slightly below the rear side. In addition, FIG. 5A illustrates a state where the insertion end 19 is passed through the through-hole 12 and the left end of each of the engaged portions 13, 14 remains close to the periphery of the through-hole 12, similarly to FIG. 2, whereas FIG. 5B illustrates a state where the insertion end 19 is deeply inserted by sliding and moving rightward with respect to the through-hole 12. In addition, the insertion end 19 can slide leftward and return from the state in FIG. 5B. In the high shape-retention retractor 11, the position of the insertion end 19 with respect to the through-hole 12 can be varied in this manner, and thus the diameter of the substantially annular ring can be freely varied. Therefore, even if some force is applied from the outside or inside of the high shape-retention retractor 11 during placement, such force can be absorbed by changing the diameter.

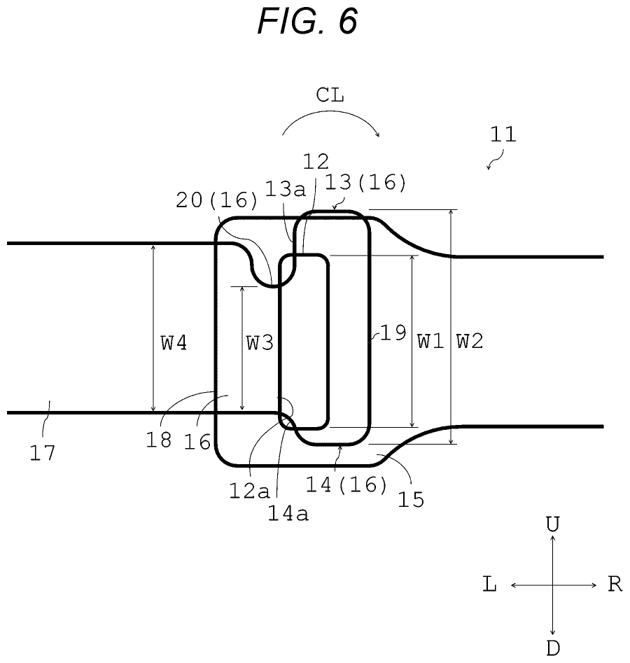

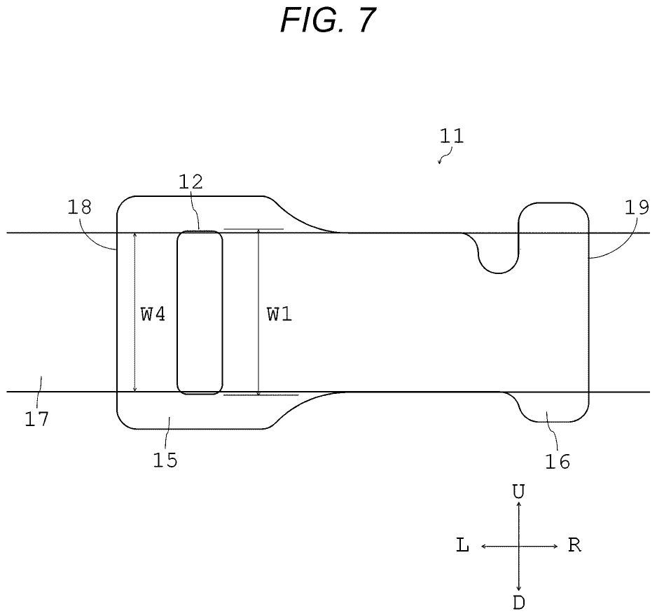

A state, where the insertion end 19 is passed through the through-hole 12, will be described in detail with reference to FIG. 6 and FIG. 7. FIG. 6 and FIG. 7 illustrate a state where the through-hole nearby portion 15 located on the right side and the engaged portion nearby portion 16 located on the left side overlap in the front-rear direction, as in FIG. 2. In front view, the insertion end 19 is hidden behind the through-hole nearby portion 15. However, areas that would originally be illustrated by hidden lines due to overlap are all illustrated by solid lines for easy understanding. FIG. 6 illustrates a state where a first engaged portion left end 13a, which is the left end of the first engaged portion 13, and a second engaged portion left end 14a, which is the left end of the second engaged portion 14, come close to a through-hole left edge 12a, which is the left side edge of the through-hole 12, in the left-right direction, similarly to FIG. 2 and FIG. 5A.

As illustrated in FIG. 6, the engaged portion nearby portion 16 has a constricted portion 20 recessed downward that is inward in the width direction. The constricted portion 20 is located adjacent to the first engaged portion 13, closer to the left. The first engaged portion 13 is formed to protrude outward in the width direction from the constricted portion 20 such that the constricted portion 20 and first engaged portion left end 13a are continuous in the up-down direction. The lower half of the constricted portion 20 is formed in a substantially semicircular shape having an arc on the lower side.

In addition, in the width direction, a through-hole width W1, which is the width of the through-hole 12, is shorter than a maximum width W2, which is a distance between the upper end of the first engaged portion 13 and the lower end of the second engaged portion 14, as illustrated in FIG. 6. Therefore, in order to pass the insertion end 19 through the through-hole 12, the engaged portion nearby portion 16 is inclined in a clockwise direction CL with respect to the through-hole nearby portion 15, the first engaged portion 13 is first passed through the through-hole 12, and then the second engaged portion 14 is passed through the through-hole 12. At this time, the constricted portion 20 is provided in the engaged portion nearby portion 16, and thus after the first engaged portion 13 is first passed through the through-hole 12, the engaged portion nearby portion 16 is brought close and upward to such an extent that the upper side edge of the through-hole 12 and the deepest portion of the constricted portion 20 come into contact with or are in close proximity to each other. Since a minimum width W3, which is a distance between the deepest portion of the constricted portion 20 and the lower end edge of the engaged portion nearby portion 16, is shorter than the through-hole width W1, the second engaged portion 14 can easily pass through the through-hole 12.

Moreover, the through-hole width W1 is shorter than the maximum width W2, and thus even when the engaged portion nearby portion 16 is pulled leftward with respect to the through-hole nearby portion 15, the first engaged portion 13, the second engaged portion 14, or both the engaged portions 13, 14 is or are engaged rightward by hooking onto the periphery of the through-hole 12. In this way, the engaged portion nearby portion 16 is engaged rightward by the through-hole nearby portion 15.

Furthermore, a normal width W4, which is a distance between the upper end and lower end of each of the engaged portion nearby portion 16 and the central portion 17 continuing leftward from the engaged portion nearby portion 16, is slightly shorter than the through-hole width W1, and thus the engaged portion nearby portion 16 and the central portion 17 on the left side of the constricted portion 20 can move left and right through the through-hole 12. At this time, the through-hole nearby portion 15, the engaged portion nearby portion 16, and the central portion 17 overlap in the front-rear direction, and thus the engaged portion nearby portion 16 and the central portion 17 slide in the left-right direction in a state of being in contact with the periphery of the through-hole 12 in the through-hole nearby portion 15. FIG. 7 is an example of a state where the insertion end 19 advances rightward and the central portion 17 passes through the through-hole 12. The through-hole width W1, the maximum width W2, the minimum width W3, and the normal width W4 described above are longer in the order of the minimum width W3, the normal width W4, the through-hole width W1, and the maximum width W2.

The high shape-retention retractor 11 preferably contains a polyether ether ketone resin as the main resin component. A polyether ether ketone resin is a material having chemical resistance and elasticity. Since the manufacturer ships a polyether ether resin in the form of a thin plate, a maker of the high shape-retention retractor 11 shapes it into a predetermined shape by performing machining such as cutting and drilling. In addition to a polyether ether ketone resin, an engineering plastic having chemical resistance, such as a polyphenylene sulfide resin (PPS), may be used.

In addition, the thickness of the high shape-retention retractor 11 is substantially uniform as a whole, and the plate thickness is preferably within the range of 0.1 mm or more and less than 0.5 mm. The inventors have found through trial and error that when the plate thickness of the high shape-retention retractor of the present invention is 0.1 mm or more and less than 0.5 mm, the high shape-retention retractor is less likely to injure the affected area, and the annular shape is more stabilized. This is because if the high shape-retention retractor 11 is too thin, there is a high risk that the affected area or its surroundings may be injured, while if the retractor is too thick, repulsive force is too strong and the nearby portions 15, 16 do not overlap each other at a predetermined position.

With the above configuration, the high shape-retention retractor 11 is configured such that the thin plate is rolled up to form a ring, and does not form a cylindrical shape like a conventional retractor. Therefore, there is little risk that a surgical instrument may cause damage or a contact with an instrument may make the shape unstable. Furthermore, the height is small, and thus a wide field of view for observing the affected area can be secured.

In addition, the high shape-retention retractor 11 contains, as the main resin component, a synthetic resin having chemical resistance, and thus it can be cleaned using a chemical product every time it is used for surgery and can be repeatedly used. By containing the synthetic resin, it is easy to mold into a thin plate shape. For example, when a polyether ether ketone resin is employed as the main resin component, the polyether ether ketone resin has heat resistance in addition to chemical resistance, and thus the retractor can be cleaned using a liquid such as a high-temperature detergent and washing water. Therefore, more effective cleaning can be performed. In addition, effective cleaning can be performed without applying a large mechanical force, and thus there is little risk that the surface may be damaged during the cleaning. This makes it possible to repeatedly use the high shape-retention retractor 11. Furthermore, a polyether ether ketone resin has impact resistance and abrasion resistance, and thus even if coming into contact with a metal instrument, the high shape-retention retractor 11 is less likely to be damaged. In addition, even if coming into contact with a part of a remotely operated instrument or robot in so-called robot surgery, the retractor is less likely to be damaged. These make it possible to use the high shape-retention retractor 11 repeatedly over a long period of time. In addition, there is no risk that the damaged portion of the retractor may injure the human body such as an internal organ.

Furthermore, the high shape-retention retractor 11 has the through-hole 12 formed in the through-hole nearby portion 15, and thus there is little risk that in a state where the insertion end 19 is passed through the through-hole 12, the through-hole nearby portion 15 may be separated from the engaged portion nearby portion 16. Therefore, the shape during use is stabilized. In addition, when the through-hole nearby portion 15 is in contact with the engaged portion nearby portion 16, a frictional force is generated between them. Therefore, by reducing the risk that the ring of the high shape-retention retractor 11 may rapidly expand or rapidly contract, the shape during use can also be stabilized.

Next, even when the annular high shape-retention retractor 11 is about to expand, the engaged portions 13, 14 hook onto the periphery of the through-hole 12, and thus the engaged portion nearby portion 16 is engaged by the through-hole nearby portion 15 in a direction in which the engaged portion nearby portion 16 tends to contract the ring, whereby further expansion can be prevented. As a result, the shape during use can be stabilized.

In addition, the engaged portion nearby portion 16 has the constricted portion 20, and thus the minimum width W3 is shorter than the through-hole width W1. Therefore, even if the through-hole width W1 is shorter, both the engaged portions 13, 14 can be passed through the through-hole 12. In addition, by shortening the through-hole width W1, both the engaged portions 13, 14 easily hook onto the periphery of the through-hole 12, and the engaged portion nearby portion 16 is more efficiently engaged by the through-hole nearby portion 15.

Although an example, in which the pair of engaged portions 13, 14 protrudes in the engaged portion nearby portion 16 of this embodiment, has been described, the high shape-retention retractor 11 may have only one engaged portion facing upward or downward as long as the engaged portion nearby portion 16 can be reliably engaged. In addition, although an example, in which only one constricted portion 20 is recessed into the engaged portion nearby portion 16, has been described, the high shape-retention retractor 11 may be provided with both an upper constricted portion and a lower constricted portion as long as necessary strength can be secured.

Next, with the plate thickness of the high shape-retention retractor 11 being 0.1 mm or more, there is little risk that the edge of the thin plate, when placed at the incision site, is too sharp and the affected area and its surroundings may be injured. On the other hand, with the plate thickness being less than 0.5 mm, it is possible to reduce occurrence of a state where the elastic force of the thin plate is too strong, the relative position between the through-hole nearby portion 15 and the engaged portion nearby portion 16 is not constant, and the hole nearby portion 15 and the engaged portion nearby portion 16 are misaligned in the up-down direction, the front-rear direction, or the left-right direction.

As a result, the high shape-retention retractor 11 can easily stabilize the shape during use.

### [Modifications]

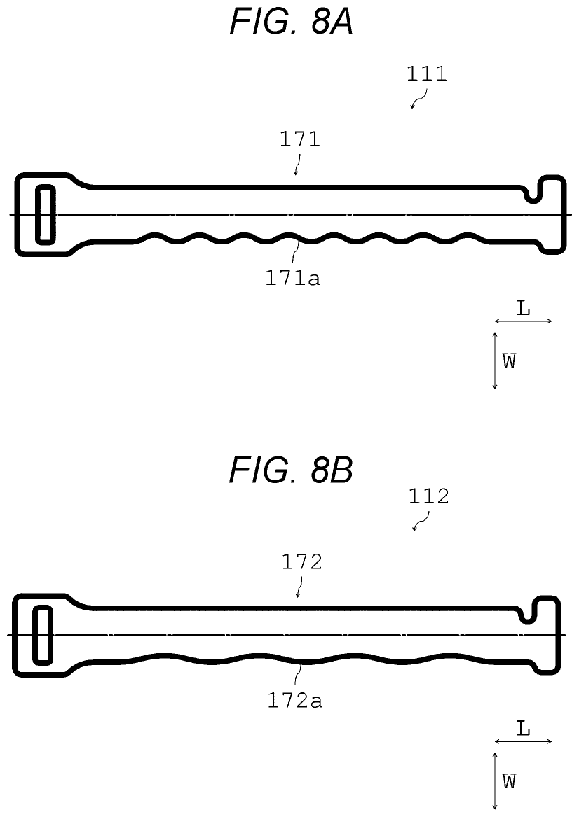

Modifications of the first embodiment of the present invention will be exemplified with reference to FIG. 8 to FIG. 10. Unless otherwise stated, the configurations of high shape-retention retractors 111 to 115 in these modifications are the same as the configuration of the high shape-retention retractor 11 described above.

First and second modifications are illustrated in FIG. 8A and FIG. 8B, respectively. In the first modification, of upper and lower side edges in a central area 171 of the high shape-retention retractor 111, the lower side edge has a wavy portion 171a. The wavy portion 171a may be located on the lower side when the high shape-retention retractor 111 is used. Therefore, when the high shape-retention retractor 111, which is placed in an upside-down orientation relative to FIG. 8A, is used, the wavy portion 171a may be provided at the side edge on the side opposite to the view. In addition, the high shape-retention retractor 111 may have the wavy portions 171a at both the upper and lower side edges in FIG. 8A. Also, in the second modification in FIG. 8B, a high shape-retention retractor 112 similarly has an wavy portion 172a in a central area 172.

The wavy portions 171a, 172a in the first and second modifications are wavy in the width directions of the high shape-retention retractors 111, 112, respectively. In the high shape-retention retractor 112 in the second modification, the pitch of the wavy portion 172a is longer than the pitch of the wavy portion 171a in the first modification.

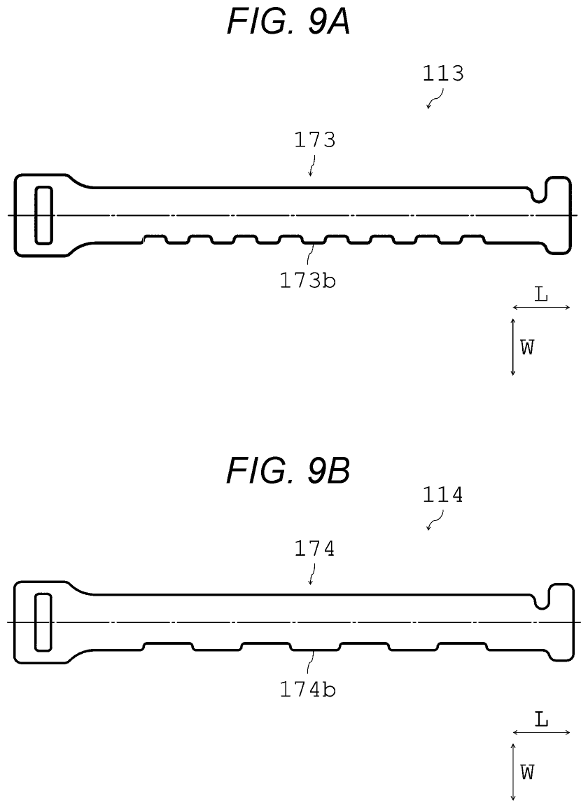

Third and fourth modifications are illustrated in FIG. 9A and FIG. 9B, respectively. In the third modification, of upper and lower side edges in a central area 173 of a high shape-retention retractor 113, the lower side edge has an uneven portion 173b. The uneven portion 173b may be located on the lower side when the high shape-retention retractor 113 is used. Therefore, when the high shape-retention retractor 113, which is placed in an upside-down orientation relative to FIG. 9A, is used, the uneven portion 173b may be provided at the side edge on the side opposite to the view. In addition, the high shape-retention retractor 113 may have the uneven portions 173b at both the upper and lower side edges in FIG. 9A. Also, in the fourth modification in FIG. 9B, a high shape-retention retractor 114 similarly has an uneven portion 174b in a central area 174.

The uneven portions 173b, 174b in the third and fourth modifications are uneven in the width directions of the high shape-retention retractors 113, 114, respectively. In the high shape-retention retractor 114 in the fourth modification, the pitch of the uneven portion 174b is longer than the pitch of the uneven portion 173b in the third modification.

Note that the corners and edges of the uneven portions 173b, 174b may be rounded to avoid injuring the affected area or its surroundings.

With the configurations in the first to fourth modifications described above, the high shape-retention retractors 111 to 114 have the wavy portions 171a, 172a or the uneven portions 173b, 174b on at least one side edge, and thus by inserting the high shape-retention retractors 111 to 114 into the incision site shown, for example, in FIG. 4 such that the side edge having the wavy portion 171a, 172a or the uneven portion 173b, 174b is located on the back side with respect to the incision site, the side edge having the wavy portion 171a, 172a or the uneven portion 173b, 174b can be in contact with the affected area or its surroundings on the back side. At this time, the wavy portions 171a, 172a are wavy or the uneven portions 173b, 174b are uneven, and thus the side edges having these are less prone to slipping against the affected area or its surroundings. Therefore, even when the high shape-retention retractors 111 to 114, which are configured to form a ring, are about to further expand outward or shrink inward, the rapid expansion or contraction can be prevented by a resistance force that the wavy portions 171a, 172a or the uneven portions 173b, 174b receive from the affected area or its surroundings. This makes it possible to stabilize the shapes of the high shape-retention retractors 111 to 114 in a moderately expanded state.

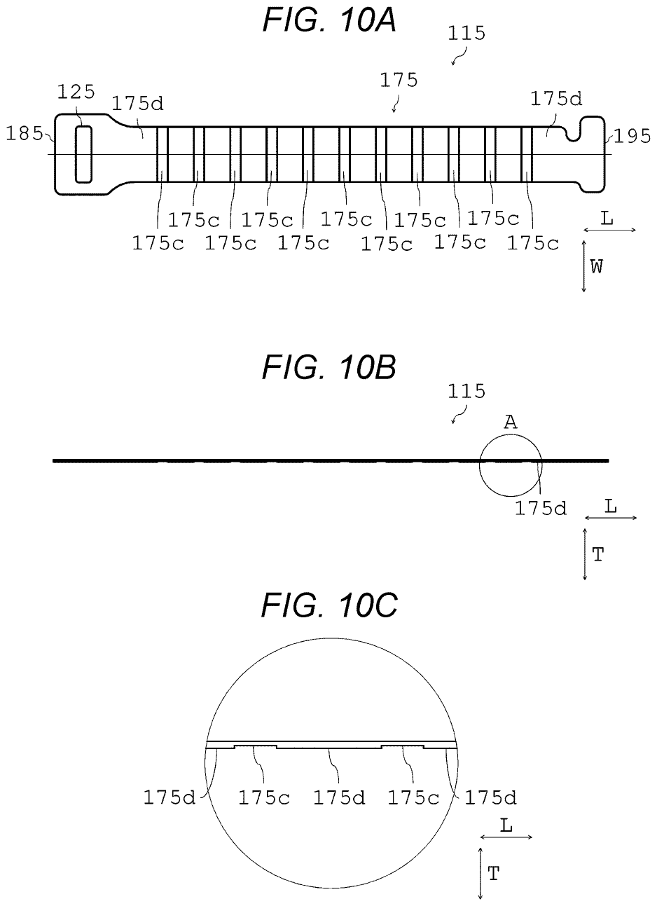

A fifth modification is illustrated in FIGS. 10A to C. A vertical arrow T illustrated in FIG. 10B and FIG. 10C indicates the thickness direction of a high shape-retention retractor 115. FIG. 10A and FIG. 10B are the front view and plan view of the high shape-retention retractor 115 in an unfolded state, respectively, while FIG. 10C is a partially enlarged plan view of an area A in FIG. 10B. In the fifth modification, the high shape-retention retractor 115 in an unfolded state has a plurality of substantially rectangular quadrilateral portions 175c recessed in a reverse direction in front of a central area 175. The high shape-retention retractor 115 can be assembled such that it is curved so as to turn, for example, an insertion end 195 to the back side and then the insertion end 195 is passed through a through-hole 182 to form a ring. The outer surface in the assembled state has the respective quadrilateral portions 175c recessed radially inward. The partial enlarged plan view of FIG. 10C illustrates a state where the quadrilateral portions 175c are recessed into a surface 175d of the central area 175 in the unfolded state.

Although not illustrated, respective quadrilateral portions 175c′ may be projected on a surface 175d′ of a central area 175′ in an unfolded state. With this, the respective quadrilateral portions 175c′ are projected radially outward in the assembled state.

With the configuration of the fifth modification described above, the high shape-retention retractor 115 has the plurality of quadrilateral portions 175c, 175c′ on the outer surface when curved, and thus when the high shape-retention retractor 115 is inserted into the incision site as shown, for example, in the photograph of FIG. 3 or FIG. 4, the quadrilateral portions 175c, 175c′ can be in contact with the affected area or the region, such as surrounding visceral organs, on the back side. At this time, the quadrilateral portions 175c, 175c′ are recessed or projected on the outer surface, and thus the central area 175, having large friction with these regions, is less prone to slipping. Therefore, even when the ring of the high shape-retention retractor 115 is about to expand or about to rotate or move, these can be prevented by the resistance force that the quadrilateral portions 175c, 175c′ receive from the affected area or the region, such as surrounding visceral organs, on the back side. This makes it possible to stabilize the shape, posture, or position of the high shape-retention retractor 115.

Note that a plurality of types of the wavy portions 171a, 172a, the uneven portions 173b, 174b, and the quadrilateral portions 175c, which have been exemplified in the first to fifth modifications, may be provided for one high shape-retention retractor. For example, the wavy portion 171a may be provided at one side edge, and the wavy portion 172a, having a longer pitch, may be provided at the other side edge. In addition, the wavy portions 171a, 172a may be provided at one side edge, and the uneven portions 173b, 174b may be provided at the other side edge. Furthermore, the quadrilateral portions 175c may be provided on the surface, and the wavy portions 171a, 172a or the uneven portions 173b, 174b may be provided at the side edges.

### Second Embodiment

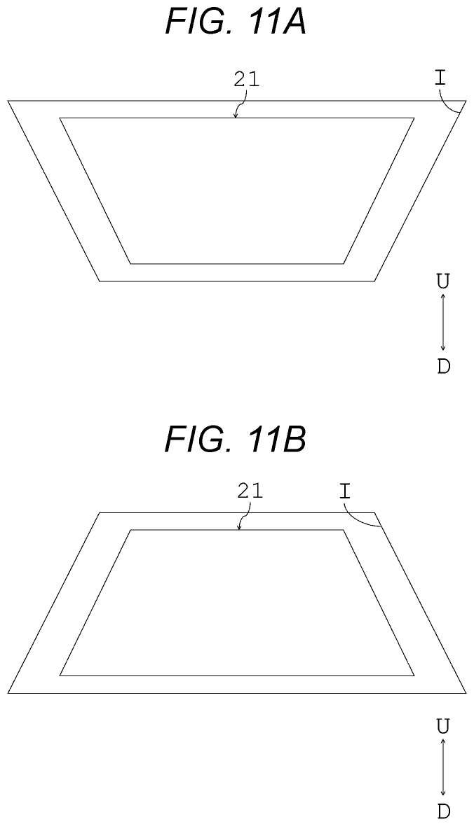

A second embodiment of the present invention will be exemplified with reference to FIG. 11 to FIG. 15. The schematic views of FIG. 11A and FIG. 11B respectively illustrate a high shape-retention retractor 21 in a state of being fitted within a region I, such as surrounding visceral organs, having an incision site in its upper portion. In FIG. 11A and FIG. 11B, the direction of an arrow U indicates the upper side of the region I such as surrounding visceral organs, and the direction of an arrow D indicates the lower side thereof. The region I, such as surrounding visceral organs, has a substantially truncated cone-shaped space whose diameter decreases toward the lower side as illustrated, for example, in FIG. 11A, or has a substantially truncated cone-shaped space whose diameter increases toward the lower side as illustrated in FIG. 11B. Alternatively, a space obtained by combining a plurality of these substantially truncated cone-shaped spaces or a space obtained by combining a substantially truncated cone-shaped space and a substantially cylindrical space is provided. In such a case, when the high shape-retention retractor 21 in its assembled state forms a part of the side surface of the substantially truncated cone, the posture of the high shape-retention retractor 21 is stabilized by the side surface extending along the inner side surface of the space of the region I such as surrounding visceral organs, as illustrated in FIG. 11A and FIG. 11B. Therefore, the high shape-retention retractor 21 can be easily formed in a substantially truncated cone shape in its assembled state as follows.

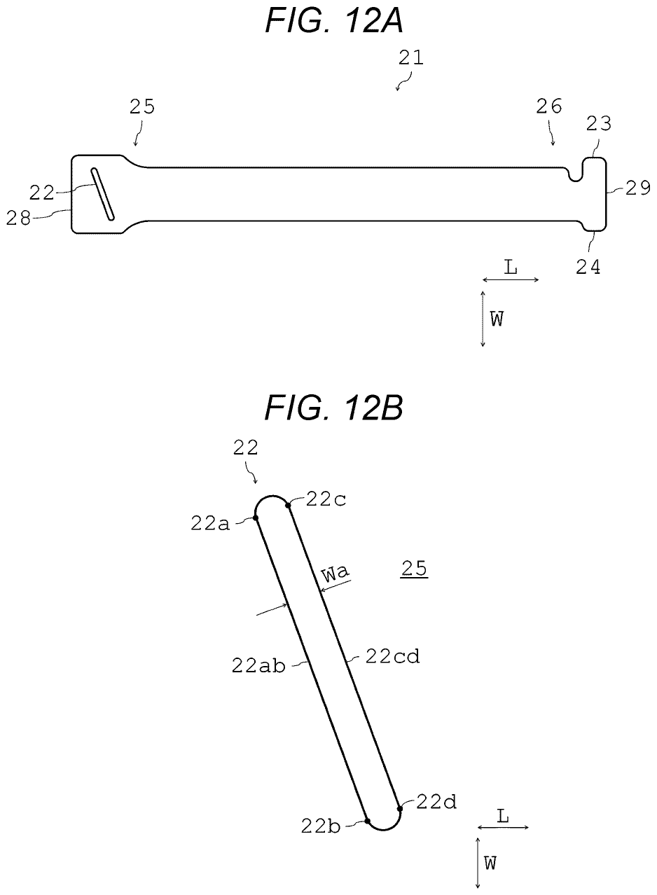

The high shape-retention retractor 21 in an unfolded state is illustrated in FIG. 12A. The horizontal arrow L illustrated in the view indicates the longitudinal direction of the high shape-retention retractor 21, while the vertical arrow W indicates the width direction thereof. Although not illustrated in the view, the high shape-retention retractor 21 has a thickness in the depth direction in the view. In the high shape-retention retractor 21, a through-hole 22 in the thickness direction is formed in the nearby area of the left end in the longitudinal direction. In addition, a first engaged portion 23 to be provided upward in FIG. 12A and a second engaged portion 24 to be provided downward therein are provided in the nearby area of the right end. The nearby area of the left end is also referred to as a through-hole nearby portion 25, while the nearby area of the right end is also referred to as an engaged portion nearby portion 26.

FIG. 12B is a partially enlarged view of the through-hole 22. The through-hole 22 has a first point 22a, which is an upper left point in FIG. 12B, and a second point 22b, which is a lower right point therein, on the side edge, on an insertion receiving end 28 side, of its inner peripheral edge. The first point 22a is disposed closer to one end in the width direction of the high shape-retention retractor 21, the one end being located in the upper side in the view, and the second point 22b is disposed closer to the other end in the width direction of the high shape-retention retractor 21, the other end being located in the lower side therein. An insertion receiving end side edge 22ab, which is the linear side edge of the through-hole 22, extends between the first point 22a and the second point 22b. That is, a line segment that is a straight line connecting the first point 22a and the second point 22b is along the insertion receiving end side edge 22ab. The line segment and the insertion receiving end side edge 22ab are inclined such that the first point 22a comes close to the left end side in the view and the second point 22b comes close to the right end side therein.

In addition, the through-hole 22 has a third point 22c, which is also an upper left point, and a fourth point 22d, which is also a lower right point, on the side edge, on an insertion end 29 side, of its inner peripheral edge. The third point 22c is disposed closer to one end in the width direction of the high shape-retention retractor 21, the one end being located in the upper side in the view, and the third point 22d is disposed closer to the other end in the width direction of the high shape-retention retractor 21, the other end being located in the lower side therein. An insertion end side edge 22cd, which is the linear side edge of the through-hole 22, extends between the third point 22c and the fourth point 22d. That is, a line segment that is a straight line connecting the third point 22c and the fourth point 22d is along the insertion end side edge 22cd. The line segment and the insertion end side edge 22ab are inclined such that the third point 22c comes close to the left end side in the view and the fourth point 22d comes close to the right end side therein. In addition, the insertion receiving end side edge 22ab and the insertion end side edge 22cd are parallel to each other.

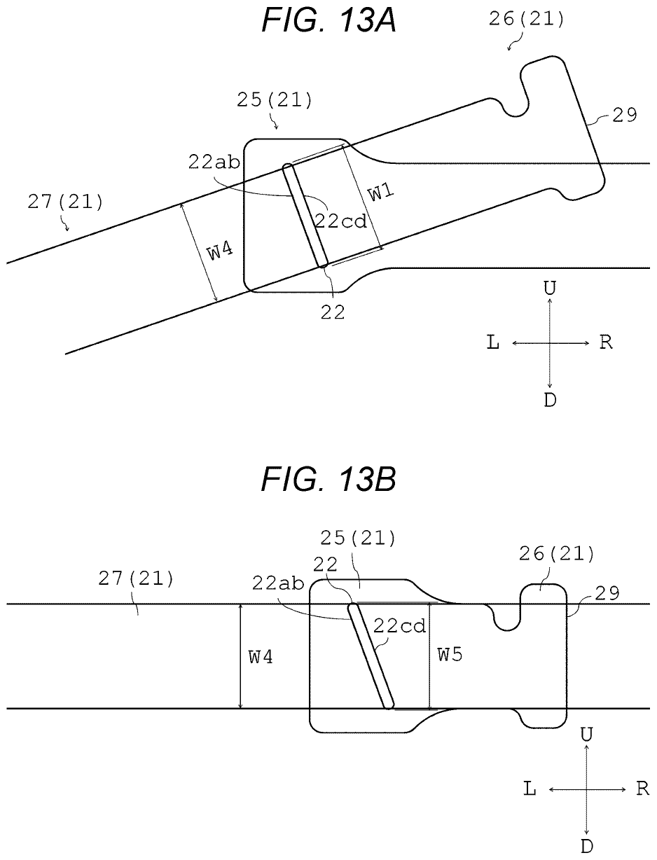

FIG. 13A and FIG. 13B illustrate an example of a state where the insertion end 29, inserted into the through-hole 22 from the front side in the view, has advanced rightward, as in FIG. 7 in the first embodiment. That is, a state is illustrated in which the engaged portion nearby portion 26 having passed through the through-hole 22 overlaps the through-hole nearby portion 25 or a central portion 27 of an area adjacent thereto on the rear side. The front side in the view is the front side of the high shape-retention retractor 21, and the back side is the rear side thereof. In addition, areas that would originally be illustrated by hidden lines due to overlap are all illustrated by solid lines for easy understanding. FIG. 13A illustrates a state where the direction of the insertion receiving end side edge 22ab coincides with the width direction of the engaged portion nearby portion 26 and the central portion 27 of the area adjacent thereto. The insertion direction is substantially orthogonal to the direction of the insertion receiving end side edge 22ab. Since the insertion receiving end side edge 22ab is inclined, the width direction of the through-hole nearby portion 25 does not coincide with the width direction of the engaged portion nearby portion 26 and the central portion 27 of the area adjacent thereto. The same applies to the insertion end side edge 22cd. This state is referred to as an orthogonal insertion state. FIG. 13B illustrates a state where the width direction of the through-hole nearby portion 25 coincides with the width direction of the engaged portion nearby portion 26 and the central portion 27 of the area adjacent thereto. The insertion direction is inclined with respect to the direction of the insertion receiving end side edge 22ab. Since the insertion receiving end side edge 22ab is inclined, the direction of the insertion receiving end side edge 22ab does not coincide with the width direction of the engaged portion nearby portion 26 and the central portion 27 of the area adjacent thereto. The same applies to the insertion end side edge 22cd. This state is referred to as an oblique insertion state.

In the orthogonal insertion state in FIG. 13A, the direction of the insertion receiving end side edge 22ab coincides with the width direction of the engaged portion nearby portion 26 and the central portion 27 of the area adjacent thereto, and the through-hole width W1, which is the width, in the direction of the insertion receiving end side edge 22ab, of the through-hole 22, is longer than the normal width W4, and thus the engaged portion nearby portion 26 and the central portion 27 of the area adjacent thereto can move in the insertion direction through the through-hole 22. In the oblique insertion state in FIG. 13B, the width direction of the through-hole nearby portion 25 coincides with the width direction of the engaged portion nearby portion 26 and the central portion 27 of the area adjacent thereto, and a right triangle height W5, which is the length, in the width direction of the high shape-retention retractor 21, of the through-hole 22 is longer than the normal width W4, and thus the engaged portion nearby portion 26 and the central portion 27 of the area adjacent thereto can move in the longitudinal direction of the high shape-retention retractor 21 through the through-hole 22. At this time, as the insertion receiving end side edge 22ab is inclined, the through-hole width W1, corresponding to the length of the oblique side of the right triangle, is longer than the right triangle height W5 corresponding to the height of the right triangle.

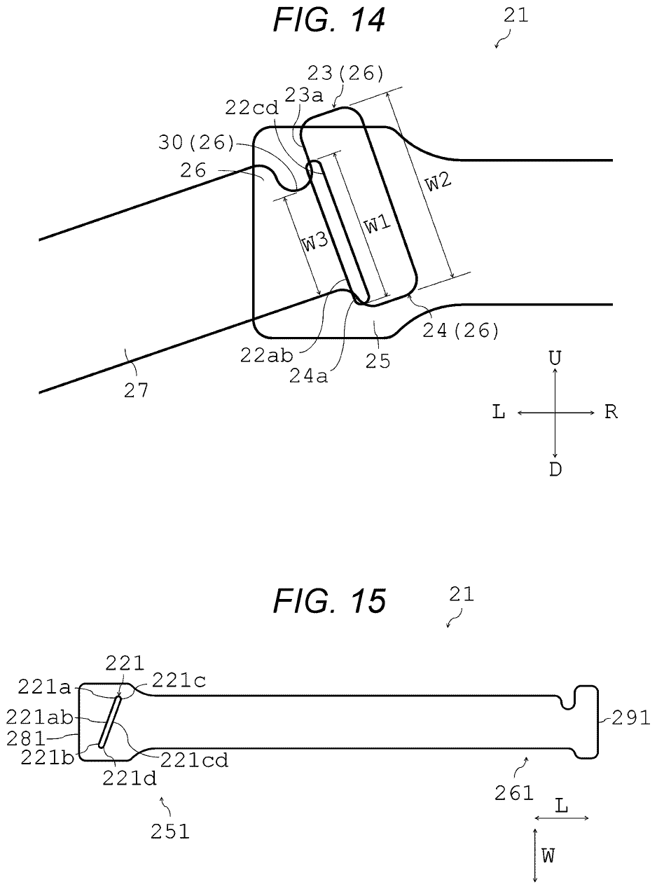

A state, where the engaged portion nearby portion 26 is pulled from the orthogonal insertion state in FIG. 13A in the direction opposite to the insertion direction, is illustrated in FIG. 14. The engaged portion nearby portion 26 is pulled in the lower left direction in the view. At this time, the through-hole width W1 is shorter than the maximum width W2, and thus a first engaged portion left end 23a of the first engaged portion 23 and a second engaged portion left end 24a of the second engaged portion 24 come close to or into contact with the insertion receiving end side edge 22ab. Therefore, even in such a pulled state, the engaged portions 23, 24 are engaged in the insertion direction by hooking onto the periphery of the through-hole 22.

Other configurations are the same as those of the first embodiment.

With the above configurations, the outward-facing surface of the high shape-retention retractor 21 forms, in the oblique insertion state, the side surface of a substantially disk-shaped body having a vertical center line. In addition, in the high shape-retention retractor 21, the extension lines of the respective generatrices at different positions in the length direction intersect with or come close to each other below the high shape-retention retractor 21 in the orthogonal insertion state, and the outward-facing surface forms a part of the side surface of a substantially truncated cone having a substantially vertical center line and having a diameter that decreases toward the lower side. As described above, when the outward-facing surface of the high shape-retention retractor 21 forms a part of the side surface of the substantially truncated cone, this surface can follow the shape of a part or the whole of the space of the site such as surrounding internal organs.

For example, when the through-hole width W1, which is the length of the through-hole 22 in the inclination direction of the insertion receiving end side edge 22ab, is not much larger than the normal width W4, the engaged portion nearby portion 26 and the central portion 27 of the area adjacent thereto are less likely to be caught on the periphery of the through-hole 22 and are easier to pass through when passing through the through-hole 22 in the direction orthogonal to the inclination direction in the orthogonal insertion state, than when passing through the through-hole 22 in a direction not orthogonal to the inclination direction in the oblique insertion state. This is because the through-hole width W1 is longer than the right triangle height W5. Therefore, in the high shape-retention retractor 21, the insertion receiving end side edge 22ab is inclined, and thus while the outward-facing surface in the orthogonal insertion state forms a part of the side surface of the substantially truncated cone, the diameter can be slightly varied by changing the insertion depth. As a result, it is easy for the outward-facing surface to follow the shape of a part or the whole of the space of the region such as surrounding visceral organs, and the retractor, expanded after being inserted into the incision site, fits well.

In either the orthogonal insertion state or the oblique insertion state, if a slit width Wa of the through-hole 22, the slit width being a width in the direction orthogonal to the insertion receiving end side edge 22ab, is shorter than a certain width, the engaged portion nearby portion 26 and the central portion 27 of the area adjacent thereto are even more likely to be caught on the periphery of the through-hole 22 and are difficult to pass through. This is because despite the engaged portion nearby portion 26 being inserted at a shallow angle with respect to the periphery of the through-hole 22 in surface-direction view of the high shape-retention retractor 21, the friction caused by the contact between the through-hole nearby portion 25 and the engaged portion nearby portion 26 increases as the slit width Wa decreases.

In addition, in the orthogonal insertion state and in a state where the engaged portion nearby portion 26 is pulled in the direction opposite to the insertion direction as illustrated in FIG. 14, the first engaged portion 23 and the second engaged portion 24 come close to or into contact with the insertion receiving end side edge 22ab of the through-hole 22, so that the engaged portion nearby portion 26 is engaged in the insertion direction by the through-hole nearby portion 25. The first engaged portion left end 23a and the second engaged portion left end 24a are located on a straight line in the width direction of the high shape-retention retractor 21. Therefore, when the engaged portion nearby portion 26 is pulled in the direction opposite to the insertion direction, the through-hole nearby portion 25 can engage with both the first engaged portion 23 and the second engaged portion 24 with an equal force. As a result, the high shape-retention retractor 21 can maintain the state where the engaged portion nearby portion 26 is engaged in the insertion direction while maintaining a constant substantially truncated cone shape. The high shape-retention retractor 21 is capable of maintaining a stable assembled state.

A modification of the second embodiment of the present invention will be exemplified with reference to FIG. 15. Unless otherwise stated, the configuration of a high shape-retention retractor 211 in this modification is the same as the configuration of the high shape-retention retractor 21 described above.

The inclination direction of a through-hole 221 of the high shape-retention retractor 211 with respect to the longitudinal direction is opposite to that of the high shape-retention retractor 21 described above. That is, an insertion receiving end side edge 221ab is inclined such that a first point 221a comes close to the right end side in the view, while a second point 221b comes close to the left end side in the view. In addition, an insertion end side edge 221cd is inclined such that a third point 221c comes close to the right end side in the view, while a fourth point 221d comes close to the left end side in the view.

With this configuration, in the high shape-retention retractor 211, the extension lines of the respective generatrices at different positions in the length direction intersect with or come close to each other below the high shape-retention retractor 211 in the orthogonal insertion state, and the outward-facing surface forms a part of the side surface of a substantially truncated cone having a substantially vertical center line and having a diameter that decreases toward the upper side.

In the high shape-retention retractors 21, 211 exemplified in this embodiment, the state where the insertion end 29, 291 is inserted into the through-hole 22, 221 may only be in the orthogonal insertion state. In this case, however, the outward-facing surface always forms a part of the side surface of the substantially truncated cone. That is, although the through-hole width W1 is larger than the normal width W4, due to the right triangle height W5 being the same or shorter, an oblique insertion state, where the width direction of the through-hole nearby portion 25, 251 coincides with the width direction of the engaged portion nearby portion 26, 261, cannot be achieved. Therefore, the high shape-retention retractors 21, 211 cannot form the side surface of the disk-shaped body.

### Third Embodiment

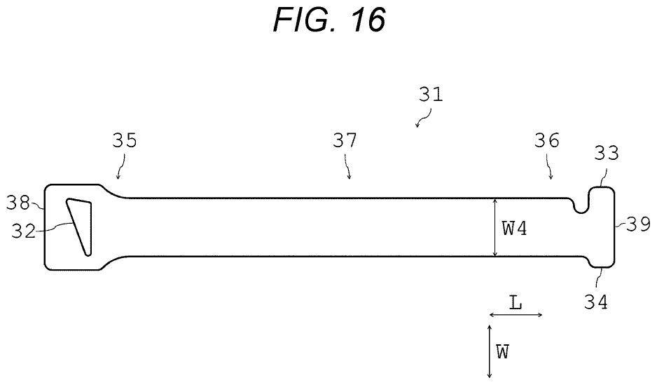

A third embodiment of the present invention will be exemplified with reference to FIG. 16 to FIG. 19. FIG. 16 illustrates a high shape-retention retractor 31 in an unfolded state. In the high shape-retention retractor 31, a through-hole 32 in the thickness direction is formed in the nearby area of the left end in the longitudinal direction. In addition, a first engaged portion 33 to be provided upward in the view and a second engaged portion 34 to be provided downward therein are provided in the nearby area of the right end. The nearby area of the left end is also referred to as a through-hole nearby portion 35, while the nearby area of the right end is also referred to as an engaged portion nearby portion 36.

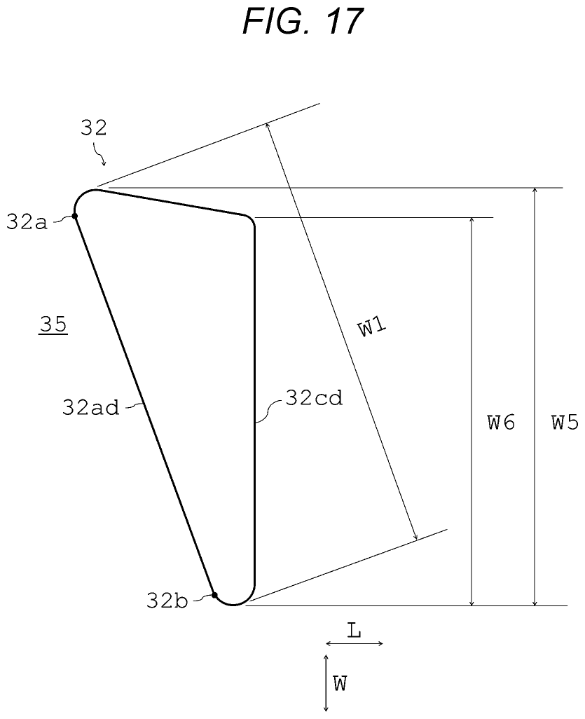

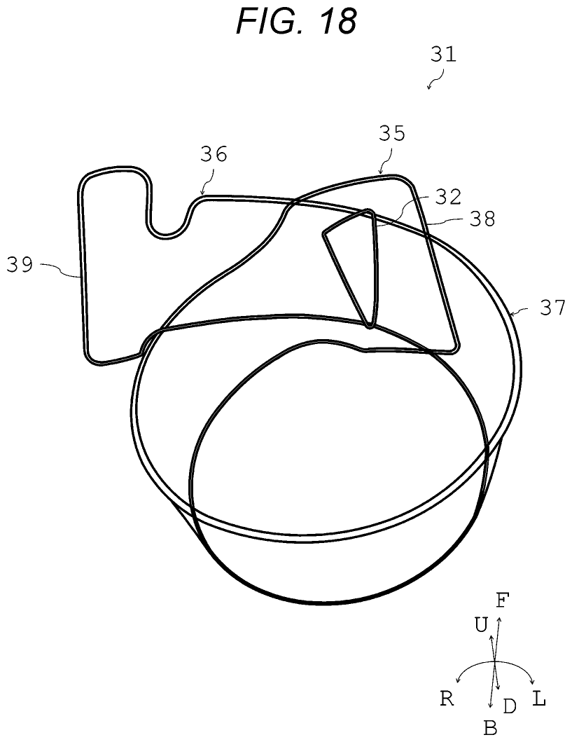

FIG. 17 is a partially enlarged view of the through-hole 32. The through-hole 32 has a first point 32a, which is an upper left point in FIG. 17, and a second point 32b, which is a lower right point therein, on the side edge, on an insertion receiving end 38 side, of its inner peripheral edge. The first point 32a is disposed closer to one end in the width direction of the high shape-retention retractor 31, the one end being located in the upper side in the view, and the second point 32b is disposed closer to the other end in the width direction of the high shape-retention retractor 31, the other end being located in the lower side therein. An insertion receiving end side edge 32ab, which is the linear side edge of the through-hole 32, extends between the first point 32a and the second point 32b. That is, a line segment that is a straight line connecting the first point 32a and the second point 32b is along the insertion receiving end side edge 32ab. The line segment and the insertion receiving end side edge 32ab are inclined such that the first point 32a comes close to the left end side in the view and the second point 32b comes close to the right end side therein. W5 is the same length as the height of the right triangle in the second embodiment, and is referred to as a right triangle height as in the second embodiment.

FIG. 18 illustrates an example of a state where, in the assembled state of the high shape-retention retractor 31, an insertion end 39 inserted into the through-hole 32 from the front side of the high shape-retention retractor 31 on the back side in the view has advanced rightward of the high shape-retention retractor 31, which is leftward in the view. This view is created by tracing an image taken of a transparent model made for easy understanding. The high shape-retention retractor 31 in this view is in the orthogonal insertion state. By changing the insertion direction such that the width direction of the through-hole nearby portion 35 coincides with the width direction of the engaged portion nearby portion 36 and the central portion 37 of the area adjacent thereto, the high shape-retention retractor 31 in its assembled state can be changed to be in the oblique insertion state. In the high shape-retention retractor 31, the extension lines of the respective generatrices at different positions in the length direction intersect with or come close to each other below the high shape-retention retractor 31 in the orthogonal insertion state, and the outward-facing surface forms a part of the side surface of a substantially truncated cone having a substantially vertical center line and having a diameter that decreases toward the lower side. FIG. 18 illustrates the shape. In the oblique insertion state, the outward-facing surface forms the side surface of a substantially disk-shaped body having a vertical center line.

Note that a second through-hole width W6, which is the width of the insertion end side edge 32cd located adjacent to the insertion receiving end side edge 32ad of the through-hole 32 in FIG. 17, closer to the right, is shorter than the through-hole width W1, shorter than the normal width W4, and shorter than the right triangle height W5.

Other configurations are the same as those of the second embodiment.

In the orthogonal insertion state in FIG. 18, the direction of the insertion receiving end side edge 32ab coincides with the width direction of the engaged portion nearby portion 36 and the central portion 37 of the area adjacent thereto, and the through-hole width W1, which is the width, in the direction of the insertion receiving end side edge 32ab, of the through-hole 32, is longer than the normal width W4, and thus the engaged portion nearby portion 36 and the central portion 37 of the area adjacent thereto can move in the insertion direction through the through-hole 32. Although not illustrated, the width direction of the through-hole nearby portion 35 coincides with the width direction of the engaged portion nearby portion 36 and the central portion 37 of the area adjacent thereto in the oblique insertion state, and the right triangle height W5, which is the length, in the width direction of the high shape-retention retractor 31, of the through-hole 32, is longer than the normal width W4, and thus the engaged portion nearby portion 36 and the central portion 37 of the area adjacent thereto can move in the longitudinal direction of the high shape-retention retractor 31 through the through-hole 32. At this time, as the insertion receiving end side edge 32ab is inclined, the through-hole width W1 is longer than the right triangle height W5.

For example, when the through-hole width W1 is not much larger than the normal width W4, the engaged portion nearby portion 36 and the central portion 37 of the area adjacent thereto are less likely to be caught on the periphery of the through-hole 32 and are easier to pass through when passing through the through-hole 32 in the direction orthogonal to the inclination direction in the orthogonal insertion state, than when passing through the through-hole 32 in a direction not orthogonal to the inclination direction in the oblique insertion state. This is because the through-hole width W1 is longer than the right triangle height W5. Therefore, in the high shape-retention retractor 31, the insertion receiving end side edge 32ab is inclined, and thus while the outward-facing surface in the orthogonal insertion state forms a part of the side surface of the substantially truncated cone, the diameter can be slightly varied by changing the insertion depth. As a result, it is easier for the outward-facing surface to follow the shape of a part or the whole of the space of the region such as surrounding visceral organs, and the retractor fits well after being inserted into the incision site.

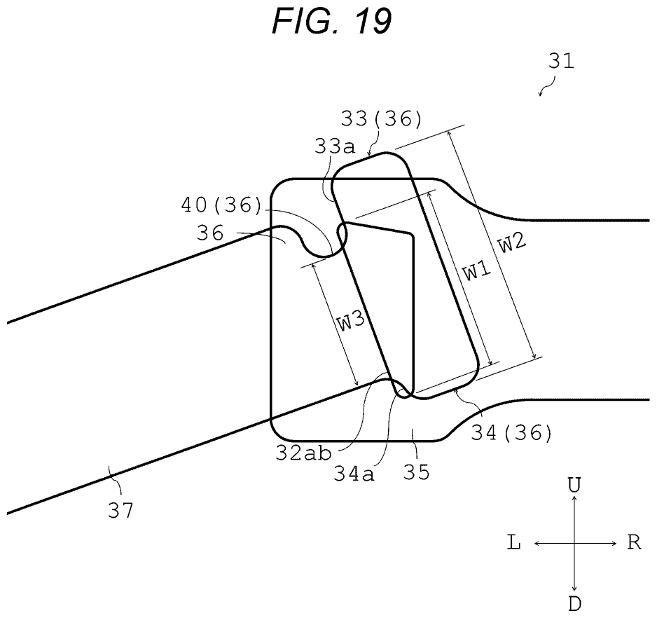

A state, where the engaged portion nearby portion 36 is pulled from the orthogonal insertion state in FIG. 18 in the direction opposite to the insertion direction, is illustrated in FIG. 19. The engaged portion nearby portion 36 is pulled in the lower left direction in the view. At this time, the through-hole width W1 is shorter than the maximum width W2, and thus a first engaged portion left end 33a of the first engaged portion 33 and a second engaged portion left end 34a of the second engaged portion 34 come close to or into contact with the insertion receiving end side edge 32ab. Therefore, even in such a pulled state, the engaged portions 33, 34 are engaged in the insertion direction by hooking onto the periphery of the through-hole 32, and as a result, the engaged portion nearby portion 36 is engaged in the insertion direction by the through-hole nearby portion 25.

The first engaged portion left end 33a and the second engaged portion left end 34a are located on a straight line in the width direction of the high shape-retention retractor 31. Therefore, when the engaged portion nearby portion 36 is pulled in the direction opposite to the insertion direction, the through-hole nearby portion 35 can engage with both the first engaged portion 33 and the second engaged portion 34 with an equal force. As a result, the high shape-retention retractor 31 can maintain the state where the engaged portion nearby portion 36 is engaged in the insertion direction while maintaining a constant substantially truncated cone shape. The high shape-retention retractor 31 is capable of maintaining a stable assembled state.

Note that in the high shape-retention retractor 31 exemplified in this embodiment, the state where the insertion end 39 is inserted into the through-hole 32 may only be in the orthogonal insertion state. In this case, however, the outward-facing surface always forms a part of the side surface of the substantially truncated cone. That is, although the through-hole width W1 is larger than the normal width W4, due to the right triangle height W5 being the same or shorter, an oblique insertion state, where the width direction of the through-hole nearby portion 35 coincides with the width direction of the engaged portion nearby portion 36, cannot be achieved. Therefore, the high shape-retention retractor 31 cannot form the side surface of the disk-shaped body.

### Fourth Embodiment

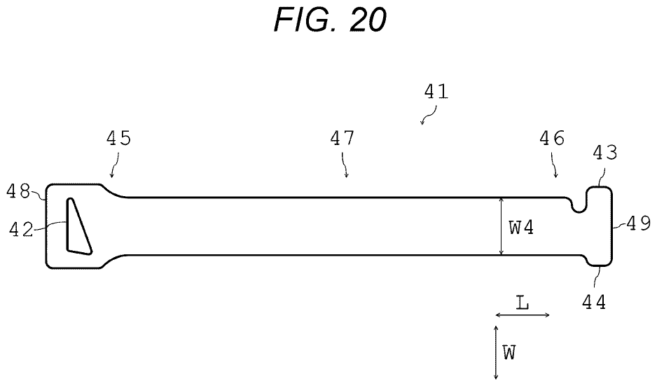

A fourth embodiment of the present invention will be exemplified with reference to FIG. 20 to FIG. 23. FIG. 20 illustrates a high shape-retention retractor 41 in an unfolded state. In the high shape-retention retractor 41, a through-hole 42 in the thickness direction is formed in the nearby area of the left end in the longitudinal direction. In addition, a first engaged portion 43 to be provided upward in the view and a second engaged portion 44 to be provided downward therein are provided in the nearby area of the right end. The nearby area of the left end is also referred to as a through-hole nearby portion 45, while the nearby area of the right end is also referred to as an engaged portion nearby portion 46.

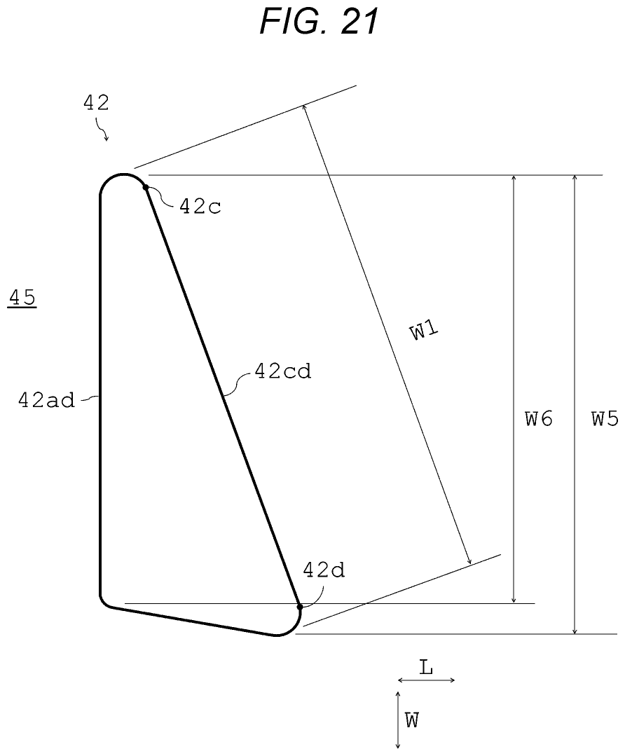

FIG. 21 is a partially enlarged view of the through-hole 42. The through-hole 42 has a first point 42c, which is an upper left point in FIG. 21, and a second point 42d, which is a lower right point therein, on the side edge, on an insertion end 49 side, of an inner peripheral edge, the side edge being located on the right side in FIG. 20. The first point 42c is disposed closer to one end in the width direction of the high shape-retention retractor 41, the one end being located in the upper side in the view, and the second point 42d is disposed closer to the other end in the width direction of the high shape-retention retractor 41, the other end being located in the lower side therein. An insertion end side edge 42cd, which is the linear side edge of the through-hole 42, extends between the first point 42c and the second point 42d. That is, a line segment that is a straight line connecting the first point 42c and the second point 42d is along the insertion end side edge 42cd. The line segment and the insertion end side edge 42cd are inclined such that the first point 42c comes close to the left end side in the view and the second point 42d comes close to the right end side therein. W5 is the same length as the height of the right triangle in the second embodiment and the third embodiment, and this is referred to as a right triangle height as in the second embodiment. In FIG. 21, an insertion receiving end side edge 42ab located adjacent to the insertion end side edge 42cd, closer to the left, is formed in a substantially linear shape in the width direction of the high shape-retention retractor 41.

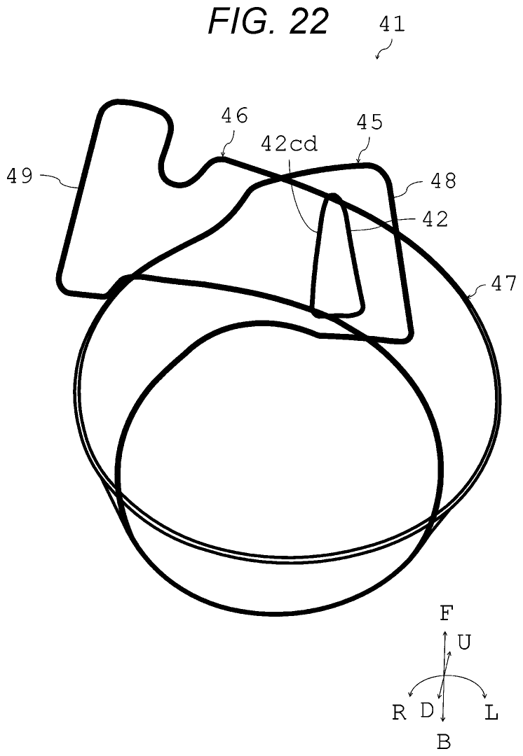

FIG. 22 illustrates an example of a state where, in the assembled state of the high shape-retention retractor 41, an insertion end 49 inserted into the through-hole 42 from the front side of the high shape-retention retractor 41 on the back side in the view has advanced rightward of the high shape-retention retractor 41, which is leftward in the view. This view is created by tracing an image taken of a transparent model made for easy understanding. The insertion direction is substantially orthogonal to the direction of the insertion end side edge 42cd. The state of the high shape-retention retractor 41 in this view is referred to as an orthogonal insertion state. In addition, the insertion direction can be changed to a direction in which the width direction of the through-hole nearby portion 45 coincides with the width direction of the engaged portion nearby portion 46 and the central portion 47 of the area adjacent thereto. This state is referred to as an oblique insertion state similarly to the second and third embodiments. In the high shape-retention retractor 41, the extension lines of the respective generatrices at different positions in the length direction intersect with or come close to each other below the high shape-retention retractor 41 in the orthogonal insertion state, and the outward-facing surface forms a part of the side surface of a substantially truncated cone having a substantially vertical center line and having a diameter that decreases toward the lower side. FIG. 22 illustrates the shape. In the oblique insertion state, the outward-facing surface forms the side surface of a substantially disk-shaped body having a vertical center line.

The second through-hole width W6, which is the width of the insertion receiving end side edge 42ab, is shorter than the through-hole width W1, shorter than the normal width W4, and shorter than the right triangle height W5.

Other configurations are the same as those of the third embodiment.

In the orthogonal insertion state in FIG. 22, the direction of the insertion end side edge 42cd substantially coincides with the width direction of the engaged portion nearby portion 46 and the central portion 47 of the area adjacent thereto, and the through-hole width W1, which is the width, in the direction of the insertion end side edge 42cd, of the through-hole 42, is longer than the normal width W4, and thus the engaged portion nearby portion 46 and the central portion 47 of the area adjacent thereto can move in the insertion direction through the through-hole 42. Although not illustrated, the width direction of the through-hole nearby portion 45 coincides with the width direction of the engaged portion nearby portion 46 and the central portion 47 of the area adjacent thereto in the oblique insertion state, and the right triangle height W5, which is the length, in the width direction of the high shape-retention retractor 41, of the through-hole 42, is longer than the normal width W4, and thus the engaged portion nearby portion 46 and the central portion 47 of the area adjacent thereto can move in the longitudinal direction of the high shape-retention retractor 41 through the through-hole 42. At this time, as the insertion end side edge 42cd is inclined, the through-hole width W1 is longer than the right triangle height W5.

For example, when the through-hole width W1 is not much larger than the normal width W4, the engaged portion nearby portion 46 and the central portion 47 of the area adjacent thereto are less likely to be caught on the periphery of the through-hole 42 and are easier to pass through when passing through the through-hole 42 in the direction orthogonal to the inclination direction in the orthogonal insertion state, than when passing through the through-hole 42 in a direction not orthogonal to the inclination direction in the oblique insertion state. This is because the through-hole width W1 is longer than the right triangle height W5. Therefore, in the high shape-retention retractor 41, the insertion end side edge 42cd is inclined, and thus while the outward-facing surface in the orthogonal insertion state forms a part of the side surface of the substantially truncated cone, the diameter can be slightly varied by changing the insertion depth. As a result, it is easier for the outward-facing surface to follow the shape of a part or the whole of the space of the region such as surrounding visceral organs, and the retractor fits well after being inserted into the incision site.

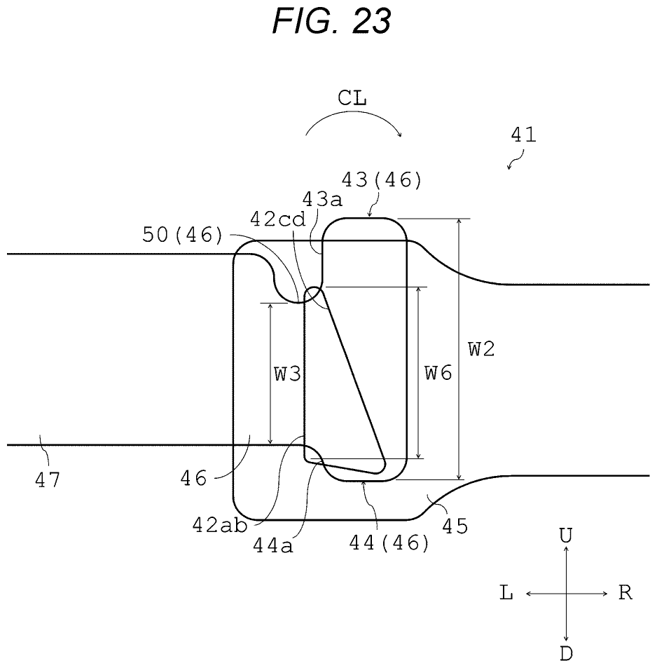

In addition, a state, where the engaged portion nearby portion 46 is pulled from the orthogonal insertion state in FIG. 22 in the direction opposite to the insertion direction, is illustrated in FIG. 23. As a result of the pulling, in the high shape-retention retractor 41 in its assembled state, the engaged portion nearby portion 46 rotates in a clockwise direction CL in the view with respect to the through-hole nearby portion 45, and the width direction of the engaged portion nearby portion 46 coincides with the width direction of the through-hole nearby portion 45 to change to the oblique insertion state. The engaged portion nearby portion 46 is pulled leftward in the view. That is, the second through-hole width W6 is shorter than the maximum width W2, and thus a first engaged portion left end 43a of the first engaged portion 43 and a second engaged portion left end 44a of the second engaged portion 44 come close to or into contact with the insertion receiving end side edge 42ab. When the second engaged portion 44 comes close to or into contact with the insertion receiving end side edge 42ab, which causes the engaged portion nearby portion 46 to stop the rotation in the clockwise direction CL. Even in such a pulled state, the engaged portions 43, 44 are engaged rightward by hooking onto the periphery of the through-hole 42, and as a result, the engaged portion nearby portion 46 is engaged rightward by the through-hole nearby portion 45.

The first engaged portion left end 43a and the second engaged portion left end 44a are located on a straight line in the width direction of the high shape-retention retractor 41. Therefore, after the engaged portion nearby portion 46 stops the rotation in the clockwise direction CL, the through-hole nearby portion 45 can engage with both the first engaged portion 43 and the second engaged portion 44 with an equal force. As a result, the high shape-retention retractor 41 can maintain the state where the engaged portion nearby portion 46 is engaged rightward while maintaining a constant substantially truncated cone shape. The high shape-retention retractor 41 is capable of maintaining a stable assembled state.

Note that in the high shape-retention retractor 41 exemplified in this embodiment, the state where the insertion end 49 is inserted into the through-hole 42 may only be in the orthogonal insertion state except for the state of being pulled in the left-right direction as illustrated in FIG. 23. In this case, however, the outward-facing surface always forms a part of the side surface of the substantially truncated cone. That is, although the through-hole width W1 is larger than the normal width W4, due to the right triangle height W5 being the same or shorter, an oblique insertion state, where the width direction of the through-hole nearby portion 45 coincides with the width direction of the engaged portion nearby portion 46, cannot be achieved. Therefore, the high shape-retention retractor 41 cannot form the side surface of the disk-shaped body.

### Fifth Embodiment

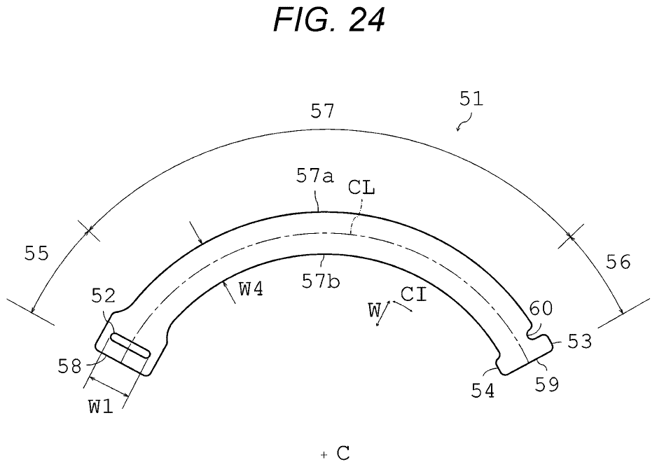

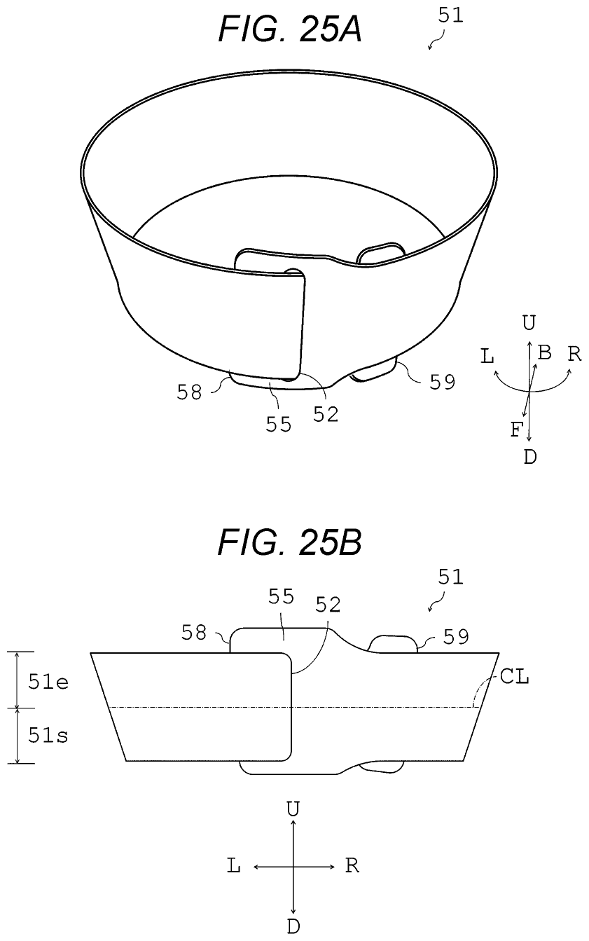

A first embodiment of the present invention will be exemplified with reference to FIG. 24 and FIG. 25. In FIG. 24, reference numeral 51 denotes a high shape-retention retractor. The high shape-retention retractor 51 of the present invention is an arc-shaped thin plate having a width in a state of being developed in a plate shape as illustrated in FIG. 24. An arrow CI illustrated in FIG. 24 indicates the circumferential direction of the high shape-retention retractor 51, while an arrow W indicates the width direction thereof. Although not illustrated in FIG. 24, the high shape-retention retractor 51 has a thickness in the depth direction in the view.

The high shape-retention retractor 51 in the deployed state has a first end that is one end in the circumferential direction and a second end that is the other end separated from the one end. The first end is located on the left side in the view, while the second end is located on the right side in the view. A through-hole 52 in the thickness direction is formed in the nearby area of the first end. In addition, a pair of engaged portions 53, 54 is provided in the nearby area of the second end. The respective engaged portions 53, 54 are provided so as to protrude toward both outward directions, respectively, in the width direction of the high shape-retention retractor 51. Of the pair of engaged portions 53, 54, the first engaged portion 53 is provided in the upper-right direction in FIG. 1, while the second engaged portion 54 is provided in the lower-left direction in FIG. 1. The first end is also referred to as an insertion receiving end 58, while the second end is also referred to as an insertion end 59 described below. Furthermore, the nearby area of the first end is also simply referred to as a first end nearby area or a through-hole nearby portion 55, while the nearby area of the second end is also simply referred to as a second end nearby area or an engaged portion nearby portion 56.

A central area that is an area sandwiched between the through-hole nearby portion 55 and the engaged portion nearby portion 56 in the circumferential direction is referred to as a central portion 57. In a state where the high shape-retention retractor 51 is developed, this central portion 57 extends in an arc shape having a width and centered on a center point C. A center line CL is provided at the midpoint in the width direction, the center line CL forming an arc centered on the center point C. Both ends 57a, 57b in the width direction of the central portion 57 also draw arcs each centered on the center point C. A normal width W4, which is a distance between an outer end 57a that is an outer end in the width direction of the central portion 57 and an inner end 57b that is an inner end therein, is slightly shorter than a passage width W1 that is the width of the through-hole 52 in the width direction.

The engaged portion nearby portion 56 has a constricted portion 60 recessed inward in the width direction. The constricted portion 60 is located adjacent to the first engaged portion 53, closer to the central portion 57.

The high shape-retention retractor 51 is elastically curved from the plate state, so that the insertion receiving end 58 side and the insertion end 59 side overlap in the thickness direction of the plate state and are rolled up, whereby a ring can be formed. FIG. 25A is a perspective view in which a state, where the insertion end 59 is curved so as to be turned to the back side from the arc-shaped state illustrated in FIG. 24 and then the insertion end 59 is passed through the through-hole 52 from the front, is viewed from obliquely above the through-hole nearby portion 55. As illustrated in this view, the insertion end 59 is passed through the through-hole 52 from the outside to the inside of the ring. FIG. 25B is a front view with the through-hole nearby portion 55 viewed from the front. The state in these views is referred to as an assembled state. The direction of an arrow U indicates the upper side in each view of the high shape-retention retractor 51 in its assembled state, while the direction of an arrow D indicates the lower side in each view thereof. The direction of an arrow L indicates the left side in each view thereof, and the direction of an arrow R indicates the right side in each view thereof. In addition, the direction of an arrow F indicates the front side in each view thereof, and the direction of an arrow B indicates the rear side in each view thereof. The outward-facing surface of the high shape-retention retractor 51 in its assembled state forms the side surface of a substantially truncated cone whose diameter increases toward the upper side. However, each arrow U, D, L, R, F, B does not necessarily indicate the upper side, lower side, left side, right side, front side, and rear side, respectively, when the high shape-retention retractor 51 is used. Furthermore, the high shape-retention retractor 51 is not only assembled by being curved so as to turn the insertion end 59 to the back side as illustrated in FIG. 2, but also assembled by being curved so as to turn the insertion end 59 from the front side in some cases, and the assembled state in this case is illustrated in a laterally reversed manner. As described above, the high shape-retention retractor 51 is used such that the direction that is convenient for a user is set to the upper side, lower side, left side, right side, front side, and rear side, and is assembled and used such that whichever surface, front or back, which is more convenient to use, is exposed to the front surface.

Other configurations are the same as those of the first embodiment.

With the above configuration, the center line CL draws an arc, and thus the high shape-retention retractor 51 forms a curved surface shape by being curved from a plate state to form a ring, and its outward-facing surface forms the side surface of the substantially truncated cone. In addition, the inward-facing surface forms the back surface of the side surface. At this time, a curved surface is formed in which a ½ area on the inner side of the center line CL, which is an arc, is inclined so as to form the ½ side surface on the small-diameter side of the substantially truncated cone. The ½ side surface on the small-diameter side is a lower half in FIG. 25B, and is indicated by reference numeral 51s in this view. The ½ side surface on the small-diameter side is referred to as a small-diameter side side surface 51s. Similarly, a curved surface is formed in which a ½ area on the outer side of the center line is inclined so as to form the ½ side surface on the large-diameter side of the substantially truncated cone. The ½ side surface on the large-diameter side is an upper half in FIG. 25B, and is indicated by reference numeral 51e in this view. The ½ side surface on the large-diameter side is referred to as a large-diameter side side surface 51e.

As a result, in the high shape-retention retractor 51 in its assembled state, it is easy for the outward-facing surface to follow the shape of a part or the whole of the space of the region such as surrounding visceral organs, and the retractor fits well after being inserted into the incision site.

In addition, the outer end 57a is expanded in the assembled state, and thus there is little risk that when the instrument is delivered to the affected area from the opening on the large-diameter side of the high shape-retention retractor 51, the instrument may be brought into contact with the outer end 57a. Therefore, it is unlikely that the instrument will damage the high shape-retention retractor 51. This is also effective in the case of robot surgery.

### Sixth Embodiment

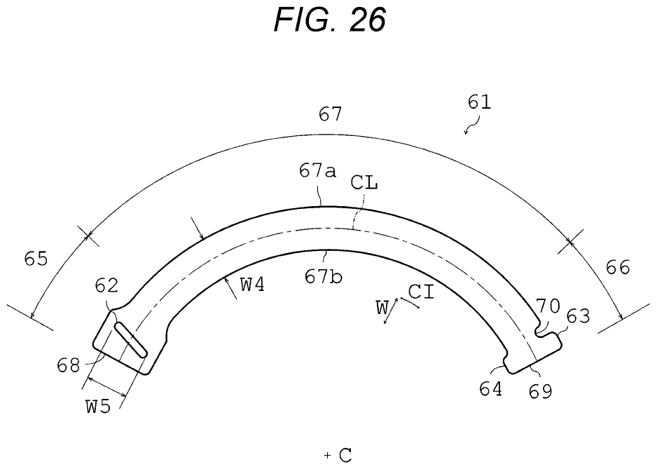

A sixth embodiment of the present invention will be exemplified with reference to FIG. 26 to FIG. 28. In FIG. 26, reference numeral 61 denotes a high shape-retention retractor. In the developed state as illustrated in FIG. 26, the high shape-retention retractor 61 has an insertion receiving end 68 and an insertion end 69. A through-hole 62 is formed in a through-hole nearby portion 65 that is the nearby portion of the insertion receiving end 68. A pair of engaged portions 63, 64 is provided in an engaged portion nearby portion 66 that is the nearby portion of the insertion end 69. The respective engaged portions 63, 64 are provided so as to protrude toward both outward directions, respectively, in the width direction of the high shape-retention retractor 61.

A normal width W4, which is a distance between an outer end 67a and an inner end 67b of a central portion 67, is slightly shorter than a right triangle height W5 that is the length, in the width direction, of the through-hole 62.

The engaged portion nearby portion 66 has a constricted portion 70 recessed inward in the width direction. The constricted portion 70 is located adjacent to the first engaged portion 63, closer to the central portion 67.

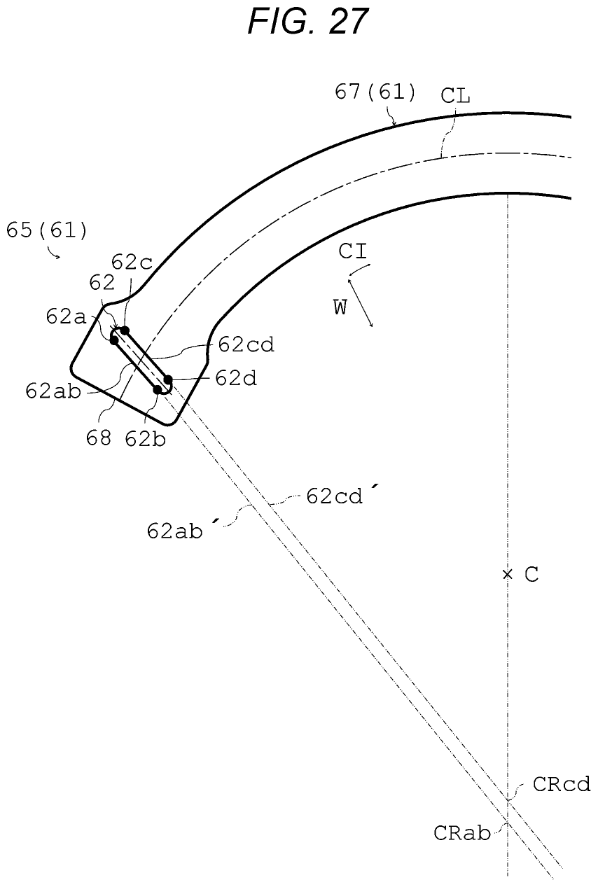

FIG. 27 is a partially enlarged view in which the through-hole nearby portion 65 of the high shape-retention retractor 61 is enlarged. The through-hole 62 has a first point 62a, which is an upper left point in FIG. 27, and a second point 62b, which is a lower right point therein, on the side edge, on an insertion receiving end 68 side, of its inner peripheral edge. The first point 62a is disposed closer to the outside of the center line CL of the high shape-retention retractor 61, and the second point 62b is disposed closer to the inside thereof. An insertion receiving end side edge 62ab, which is the linear side edge of the through-hole 62, extends between the first point 62a and the second point 62b. That is, a line segment that is a straight line connecting the first point 62a and the second point 62b is along the insertion receiving end side edge 62ab. As illustrated in FIG. 27, an inward extension line 62ab′, which is an extension line of a line segment connecting the first point 62a and the second point 62b and is an inward extension line of the arc, extends from the line segment.

The inward extension line 62ab′ passes through an intersection CRab that is farther from the arc of the center line CL than the center point C.

In addition, the through-hole 62 has a third point 62c, which is an upper left point in FIG. 27, and a fourth point 62d, which is a lower right point therein, on the side edge, on an insertion end 69 side, of its inner peripheral edge. The third point 62c is disposed closer to the outside of the center line CL of the high shape-retention retractor 61, and the fourth point 62d is disposed closer to the inside thereof. An insertion end side edge 62cd, which is the linear side edge of the through-hole 62, extends between the third point 62c and the fourth point 62d. That is, a line segment that is a straight line connecting the third point 62c and the fourth point 62d is along the insertion end side edge 62cd. As illustrated in FIG. 27, an inward extension line 62cd′, which is an extension line of a line segment connecting the third point 62c and the fourth point 62d and is an inward extension line of the arc, extends from the line segment. The inward extension line 62cd′ also passes through an intersection CRcd that is farther from the arc of the center line CL than the center point C.

In the through-hole 62, the line segment along the insertion receiving end side edge 62ab and the line segment along the insertion end side edge 62cd are both inclined with respect to the width direction of the high shape-retention retractor 61, as described above.

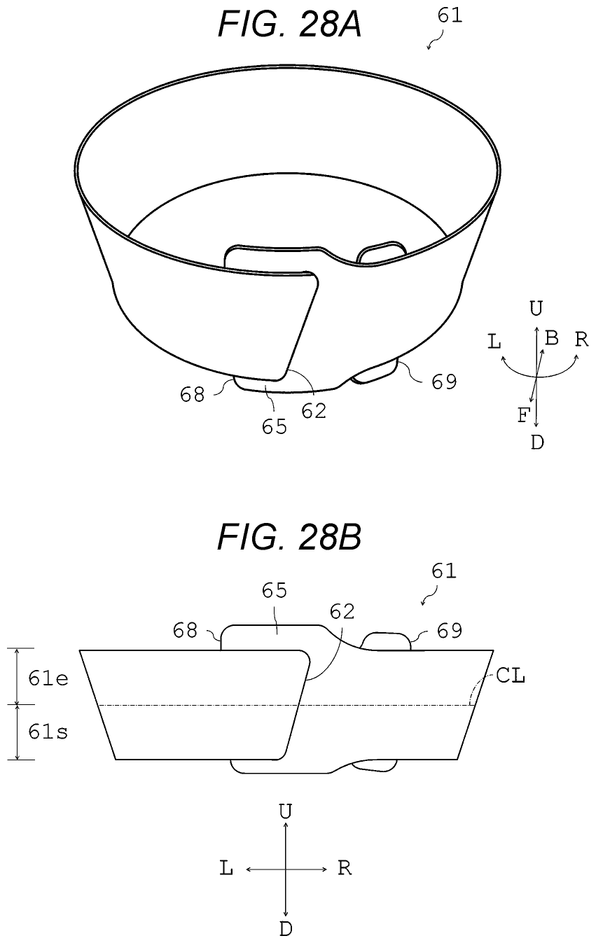

The high shape-retention retractor 61 is elastically curved from the plate state, so that the insertion receiving end 68 side and the insertion end 69 side overlap in the thickness direction of the plate state and are rolled up, whereby a ring can be formed. FIG. 28A is a perspective view in which a state, where the insertion end 69 is curved so as to be turned to the back side from the arc-shaped state illustrated in FIG. 26 and FIG. 27 and then the insertion end 69 is passed through the through-hole 62 from the front, is viewed from obliquely above the through-hole nearby portion 65. FIG. 28B is a front view with the through-hole nearby portion 65 viewed from the front.

Other configurations are the same as those of the fifth embodiment.

With the above configuration, the center line CL draws an arc, and thus the high shape-retention retractor 61 forms a curved surface shape by being curved from a plate state to form a ring, and its outward-facing surface forms the side surface of the substantially truncated cone. In addition, the inward-facing surface forms the back surface of the side surface. A ½ side surface on the small-diameter side is referred to as a small-diameter side side surface 61s, and a ½ side surface on the large-diameter side is referred to as a large-diameter side side surface 61e.

### EXAMPLES

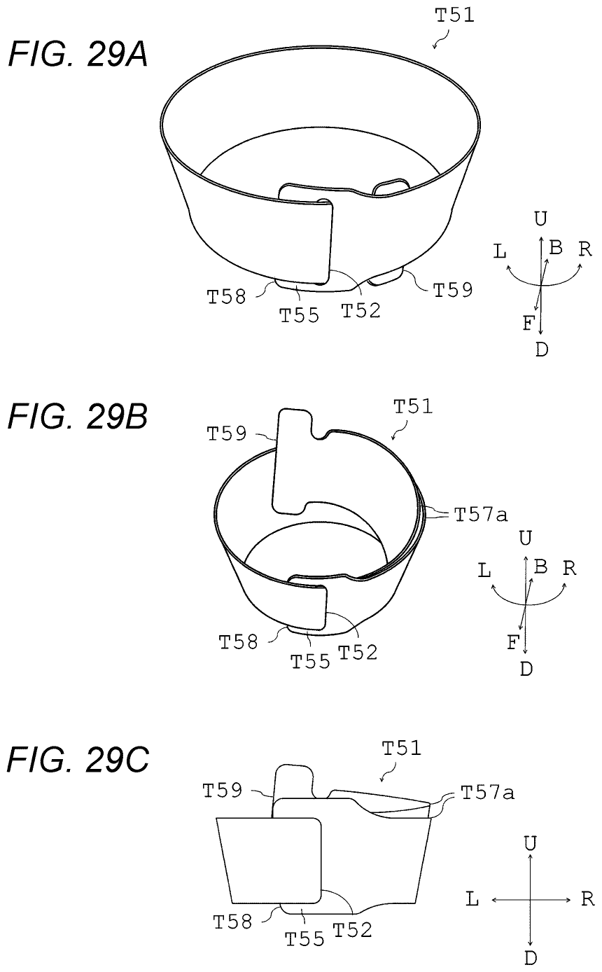

An example of the fifth embodiment will be described with reference to FIG. 29. Following the development view illustrated in FIG. 24, the inventors cut out a plate material of a polyether ether ketone resin having a plate thickness of 0.4 mm to experimentally produce the high shape-retention retractor 51 according to the fifth embodiment. The center line CL of a prototype T51, which was the prototype, had a radius of 84 mm and an arc length, which was the length of the arc of the center line CL, of 192.3 mm. The width of the central portion 57 was 16.0 mm. The length of the through-hole 52, which was an ellipse, was 16.4 mm.

FIG. 29A is a view created by tracing an image in which a state, where, similarly to FIG. 25A described above, an insertion end T59 of the prototype T51 is curved so as to be turned to the back side and then the insertion end T59 is passed through a through-hole T52 from the front, is viewed from obliquely above a through-hole nearby portion T55. FIG. 29B is a view created by tracing an image in which a state, where the insertion end T59 is further inserted from the state in FIG. 29A and rotated about a half turn from the through-hole T52, is viewed similarly from obliquely above. FIG. 29C is a view created by tracing an image in front view in which, in the state in FIG. 29B, the through-hole nearby portion T55 is viewed from the front. As illustrated in these views, it was observed that the prototype T51 had a curved surface shape by being curved from a plate state to form a ring, and its outward-facing surface formed the side surface of the substantially truncated cone, and its inward-facing surface formed the back surface of this side surface.

On the other hand, when the insertion end T59 was further rotated in this way, the inventors found that since the center line CL of the prototype T51 drew an arc, a three-dimensional shape after winding tended to form a spiral shape by the elastic force when curved from the plate shape to form a ring. Although not illustrated in the views, if the insertion end T59 was not passed through the through-hole T52, the area close to an insertion receiving end T58 and the area close to the insertion end T59, after winding, were misaligned in the up-down direction, which tended to form a spiral shape.

Then, it was found that when the insertion end T59 of such a prototype T51 was passed through the through-hole T52, the area close to the insertion end T59 tended to protrude upward in the view, which was close to the large-diameter side of the truncated cone, and as a result, there were times when the area did not overlap so as to coincide with the area close to the insertion receiving end T58 one turn earlier. In particular, such a tendency was remarkable when the winding was advanced as illustrated in FIG. 29B and FIG. 29C. In such a case, for example, an outer end T57a was gradually misaligned upward as the winding progressed. It was observed that, in this way, a force causing the insertion end T59 to be biased toward the large-diameter side of the truncated cone was applied to the prototype T51, and under this state, there were times when the outward-facing surface of the prototype T51 formed a distorted truncated cone.

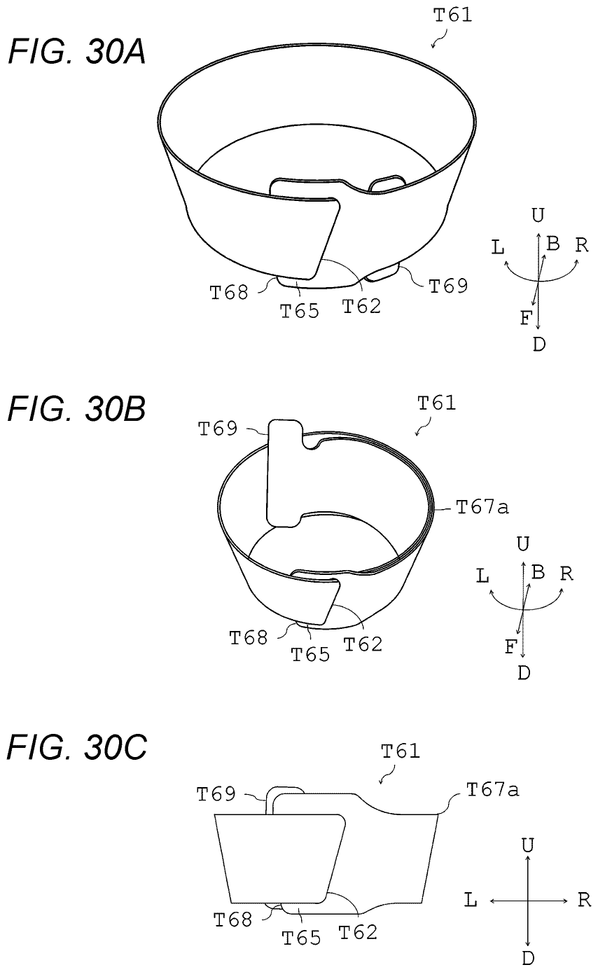

Next, an example of the sixth embodiment will be described with reference to FIG. 30. Following the development view illustrated in FIG. 26, the inventors cut out a plate material of a polyether ether ketone resin having a plate thickness of 0.4 mm to experimentally produce the high shape-retention retractor 61 according to the sixth embodiment. A center line CL of a prototype T61, which was the prototype, had a radius of 84 mm and an arc length, which was the length of the arc of the center line CL, of 192.3 mm. The width of the central portion 67 was 16.0 mm. A through-hole 62, which was an ellipse, had a length of 16.4 mm and an angle, in the length direction with respect to the width direction, of 20°.

FIG. 30A and FIG. 30B are views created by tracing an image in which the prototype T61 was viewed from obliquely above the through-hole nearby portion T65, similarly to FIG. 29A and FIG. 29B, respectively. Similarly to FIG. 29C, FIG. 30C is a view created by tracing a front view image showing the through-hole nearby portion T65 as viewed from the front. As illustrated in these views, it was observed that, similarly to the prototype T51, the prototype T61 formed a curved surface shape by being curved from a plate state to form a ring, and its outward-facing surface formed the side surface of the substantially truncated cone, and its inward-facing surface formed the back surface of this side surface.

Then, the through-hole T62 of the prototype T61 is inclined such that, in a state where the prototype T61 is developed, extension lines, extending in the arc inner direction of a line segment connecting the first point 62a and the second point 62b and a line segment connecting the third point 62c and the fourth point 62d, pass through positions farther from the arc than the center line C of the arc. Therefore, it was found that the insertion end T69 that had passed through the through-hole 62 after making a full turn tended to advance closer to the small-diameter side of the truncated cone, which is the direction orthogonal to the inclination direction, than along the arc. That is, it was found that when passing through the through-hole T62 in the direction orthogonal to the inclination direction, the insertion end T69 easily passed through, whereas when passing through in a direction not orthogonal to the inclination direction, it was likely to be caught on the periphery of the through-hole T62 and difficult to pass through the through-hole T62. Therefore, it was observed that a force that tended to bias the insertion end T69 toward the small-diameter side of the truncated cone, through which it would pass more easily, was applied to the prototype T61.

In the prototype T61, the center line CL drew an arc and the through-hole T62 was inclined in the developed state, as described above, and thus a state was able to be observed, where the force that tended to bias the insertion end T69 toward the large-diameter side of the truncated cone was balanced with the force that tended to bias it toward the small-diameter side thereof. As a result, even if the winding proceeded, the insertion end T69 was less likely to come close to either the large-diameter side or the small-diameter side, and the outward-facing surface of the prototype T61 was able to form the surface of an undistorted truncated cone, as illustrated in FIG. 30B and FIG. 30C. In addition, the inward-facing surface was able to form the back of the surface of the undistorted truncated cone. As illustrated in FIG. 30B and FIG. 30C, a situation, in which the outer end T67a was gradually misaligned upward, scarcely occurred. Therefore, it is easy for the outward-facing surface to follow the shape of a part or the whole of the space of the region such as surrounding visceral organs, and the retractor fits well after being inserted into the incision site.

Since the outer end T67a is less likely to be misaligned upward, it is possible to reduce a situation in which an instrument to be delivered to the affected area may be caught on the outer end T67a. This is also effective in the case of robot surgery.

Although the embodiments of the present invention have been exemplified above, the present invention is not limited to these embodiments, and various modifications can be made without departing from the gist of the present invention.

Although a shape, in which, for example, the insertion end is passed through the through-hole from the outside to inside of the ring, has been described, the insertion end may be passed through from the inside to outside of the ring as long as the engaged portion nearby portion in the assembled state does not greatly protrude outward of the ring. That is, the through-hole nearby portion and the engaged portion nearby portion may overlap in reverse order in the front-rear direction.

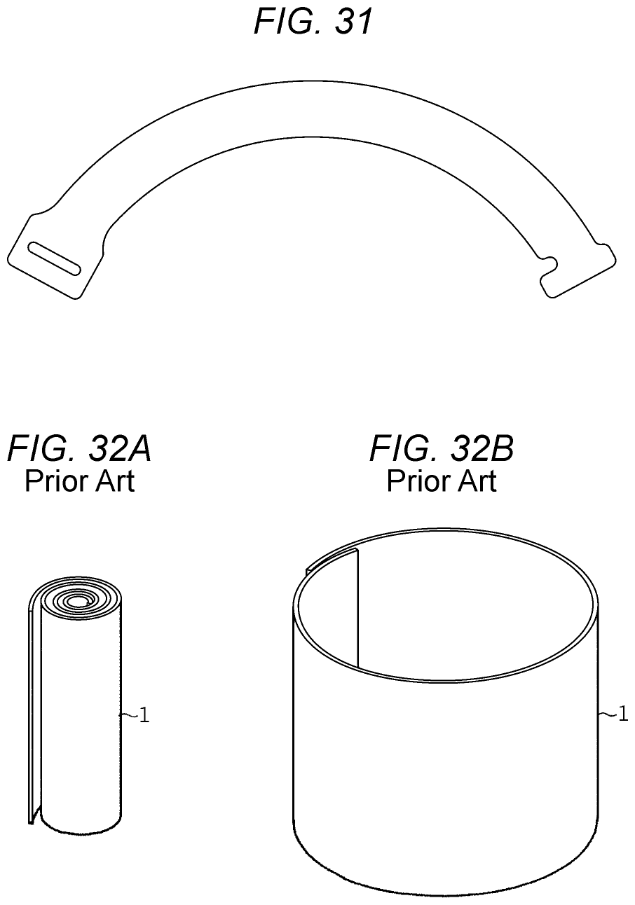

Although shapes, in which the constricted portions are recessed downward in the width direction, have been illustrated in the respective views, these constricted portions may be recessed upward in the width direction. In addition, when the high shape-retention retractors as in the fifth and sixth embodiments are formed in an arc shape, the constricted portions may be recessed adjacent to the second engaged portion, closer to the central portion, as illustrated in the example in FIG. 31. Two constricted portions may be recessed on both sides in the width direction. As described above, the high shape-retention retractor of the present invention is used such that the direction that is convenient for a user is set to the upper side, lower side, left side, right side, front side, and rear side, and it is sufficient if the constricted portion is provided in such a convenient orientation.

The contained resin component may be a resin other than the polyether ether ketone resin or the polyphenylene sulfide resin as long as it is a resin having chemical resistance.

In the fifth modification of the first embodiment, an example of the substantially rectangular quadrilateral portion has been described, however, when such a quadrilateral portion is applied to the high shape-retention retractor according to the fifth or sixth embodiment, a substantially trapezoidal quadrilateral portion may be provided instead of the substantially rectangular quadrilateral portion, as well as the formation of the high shape-retention retractor of the fifth or sixth embodiment in an arc shape having a width.

Furthermore, an example has been described in which the first engaged portion left end and the second engaged portion left end are located on a straight line in the width direction of the high shape-retention retractor, however, when the engaged portion left ends are engaged by the through-hole nearby portion in the direction in which the engaged portion nearby portions tend to contract the ring, and if these nearby portions are oriented in the intended directions, the left ends may not be located on a straight line. Then, by locating the left ends at positions other than a straight line, the ring may be expanded in the orientations of the nearby portions other than the exemplified direction.

### INDUSTRIAL APPLICABILITY

The present invention can be used for a retractor for holding an incision site of the human body to be incised in an open state.

### REFERENCE SIGNS LIST

11, 21, 31, 41, 51, 61, 111, 112, 113, 114, 115, 211 high shape-retention retractor
12, 22, 32, 42, 52, 62, 125, 221 through-hole
13, 23, 33, 43, 53, 63 first engaged portion
13a, 23a, 33a, 43a first engaged portion left end
14, 24, 34, 44, 54, 64 second engaged portion
14a, 24a, 34a, 44a second engaged portion left end
15, 25, 35, 45, 55, 65, 251 through-hole nearby portion (first end nearby area)
16, 26, 36, 46, 56, 66, 261 engaged portion nearby portion (second end nearby area)
17, 27, 37, 47, 57, 67, 171, 172, 173, 174, 175 central portion
18, 28, 38, 48, 58, 68, 185, 281 insertion receiving end (first end)
19, 29, 39, 49, 59, 69, 195, 291 insertion end (second end)
20, 30, 40, 50, 60, 70 constricted portion
22a, 32a, 42c, 62a, 221a first point
22b, 32b, 42d, 62b, 221b second point
22ab, 32ab, 42ab, 62ab, 221ab insertion receiving end side edge
22c, 62c, 221c third point
22d, 62d, 221d fourth point
22cd, 32cd, 42cd, 62cd, 221cd insertion end side edge
51e, 61e large-diameter side side surface
51s, 61s small-diameter side side surface
W1 through-hole width
W2 maximum width
W3 minimum width
W4 normal width
W5 right triangle height
W6 second through-hole width

1. A high shape-retention retractor that: contains, as a main resin component, a synthetic resin having chemical resistance and being an elastic material; has a first end and a second end separated from the first end in a state of being developed in a plate shape, and has a width direction; and is configured to be elastically curved from the plate state such that the first end and the second end overlap in a thickness direction of the plate state to form a ring, wherein: a through-hole is formed to allow the second end to pass through in a first end nearby area that is near the first end; and a length of a distance over which the second end is passed through the through-hole is adjustable to change a size of a diameter of the ring.

2. The high shape-retention retractor according to claim 1, wherein a second end nearby area that is near the second end has an engaged portion protruding from the through-hole in the width direction in a state where the second end is passed through the first end nearby area.

3. The high shape-retention retractor according to claim 2 comprising a pair of the engaged portions, wherein
the respective engaged portions protrude in both outward directions in the width direction, and
at least the engaged portion provided on one side in the width direction is formed to protrude from a constricted portion adjacent in a direction coming close to the first end and recessed inward in the width direction.

4. The high shape-retention retractor according to claim 1, wherein
an inner peripheral edge of the through-hole has a shape having a line segment between two points, and
the line segment between the two points is inclined such that, of the two points, a first point located closer to a first edge in the width direction comes closer to either the first end or the second end than a second point located closer to a second edge in the width direction does.

5. The high shape-retention retractor according to claim 1, wherein
in a central area that is an area sandwiched between the first end nearby area and the second end nearby area, a center line in the width direction draws an arc in the developed state.

6. The high shape-retention retractor according to claim 5, wherein
the inner peripheral edge of the through-hole has a shape having a line segment between two points, and the line segment between the two points is inclined with respect to the width direction such that, in the developed state, an extension line, extended inward of the arc, of the line segment between the two points passes through a position farther from the arc than a center point of the arc.

7. The high shape-retention retractor according to claim 1, wherein
a plate thickness in the thickness direction is 0.1 mm or more and less than 0.5 mm.

8. The high shape-retention retractor according to claim 1, wherein
the central area that is an area sandwiched between the first end nearby area and the second end nearby area has a wavy portion where at least one edge in the width direction is wavy in the width direction.

9. The high shape-retention retractor according to claim 1, wherein
the central area that is an area sandwiched between the first end nearby area and the second end nearby area has an uneven portion where at least one edge in the width direction is uneven in the width direction.

10. The high shape-retention retractor according to claim 1, wherein
a plurality of substantially rectangular quadrilateral portions, each having long sides in the width direction and recessed or projected in the thickness direction, are provided in the central area that is an area sandwiched between the first end nearby area and the second end nearby area, and
in a state where the ring is formed, each of the quadrilateral portions is recessed radially inward or projected radially outward of the curve on an outer surface of the curve.

11. The high shape-retention retractor according to claim 1, containing a polyether ether ketone resin as the main resin component.
